(19) United States
(12) Patent Application Publication
(10) Pub. No.: US 2025/0152591 A1
(43) Pub. Date: May 15, 2025

(54) TREATMENT OF HEMATOLOGICAL MALIGNANCIES WITH MENIN INHIBITORS AND P-GLYCOPROTEIN INHIBITORS

(71) Applicant: Kura Oncology, Inc., San Diego, CA (US)

(72) Inventors: Asako MCCLOSKEY, San Diego, CA (US); Francis BURROWS, Solana Beach, CA (US); Shivani MALIK, San Diego, CA (US); Douglas GREENE, San Clemente, CA (US); Xuefeng ZHU, San Diego, CA (US); Mollie LEONI, Dover, MA (US)

(21) Appl. No.: 18/834,563

(22) PCT Filed: Feb. 2, 2023
(86) PCT No.: PCT/US2023/061875

§ 371 Date: Jul. 30, 2024

Related U.S. Application Data
(60) Provisional application No. 63/306,901, filed on Feb. 4, 2022.

(51) Int. Cl. A61K 31/519; A61K 31/496; A61P 35/00

(52) U.S. Cl. CPC A61K 31/519; A61K 31/496; A61P 35/00

(57) ABSTRACT

The present disclosure provides methods for treating hematological malignancies using menin inhibitors in combination with P-gp inhibitors and/or BCRP inhibitors. Compositions for use in these methods are also provided.

### CROSS-REFERENCE TO RELATED APPLICATION

This application claims the benefit of U.S. Patent Application No. 63/306,901 filed Feb. 4, 2022, which is incorporated by reference in its entirety.

### BACKGROUND

The mixed-lineage leukemia (MLL) protein (also known as histone-lysine-N-methyltransferase 2A (KMT2A) protein, hereinafter, KMT2A(MLL) protein) is a histone methyltransferase critical for the epigenetic regulation of gene transcription. Many acute leukemias, including acute myeloblastic leukemia (AML), acute lymphoblastic leukemia (ALL) and mixed-lineage leukemia (MLL), are characterized by the presence of chimeric KMT2A(MLL) fusion proteins that result from chromosomal translocations of the MLL gene (also known as histone-lysine-N-methyltransferase 2A (KMT2A) gene, hereinafter KMT2A(MLL) gene) located at chromosome 11, band q23 (11q23). Chimeric MLL fusion proteins retain approximately 1,400 amino acids of the N-terminus of KMT2A(MLL) but are fused with one of approximately 80 partner proteins (e.g., AF4, AF9, ENL, AF10, ELL, AF6, AF1p, GAS7). KMT2A(MLL) fusion proteins lack the original histone methyltransferase activity of the C-terminus of KMT2A(MLL) and gain the ability to regulate transcription of numerous oncogenes, including homeobox (HOX) and myeloid ecotropic viral insertion site 1 (MEIS1), resulting in increased cell proliferation and decreased cell differentiation, ultimately leading to leukemogenesis.

The menin protein, which is encoded by the multiple endocrine neoplasia (MEN) gene, is a ubiquitously expressed nuclear protein that engages in interactions with DNA processing and repair proteins, chromatin modifying proteins and numerous transcription factors. The association of menin with the N-terminus of KMT2A(MLL) fusion proteins is necessary for the observed oncogenic activity of KMT2A(MLL) fusion proteins. This association has been shown to constitutively up-regulate the expression of HOX and MEIS1 oncogenes and impairs proliferation and differentiation of hematopoietic cells leading to leukemia development. Since menin has been shown to function as a general oncogenic cofactor in KMT2A(MLL)-related leukemias, the interaction between menin and a KMT2A(MLL) protein (either wild-type or a KMT2A(MLL) fusion protein) represents a potential therapeutic target. Patients, especially infants, with leukemias harboring chromosomal translocations of the KMT2A(MLL) gene have a dismal prognosis, with less than a 40% five-year survival rate.

P-Glycoprotein (P-gp) is a member of the ATP binding cassette (ABC) transporter family that has been a widely studied drug transporter protein (see, e.g., Balimane et al., AAPS J., 2008, 10(4):577-586). P-gp is a major determinant of absorption, distribution, and elimination of a wide variety of drugs, can modulate pharmacokinetics of drugs, and can play a pivotal role in the pharmacokinetics and distribution of drugs into target organs (see id.). Breast cancer resistance protein (BCRP) is also an ABC efflux transporter that restricts distribution of certain drugs into organs and increases elimination of substrates in the gut and biliary tract and through other bodily membranes.

### SUMMARY

Certain menin inhibitors (e.g., Compound I) are substrates of P-gp and/or a breast cancer resistance protein (BCRP), which are cellular efflux transporters that can confer resistance to chemotherapeutic agents. Cellular efflux transporter inhibitors, such as P-gp inhibitors and/or BCRP inhibitors, can enhance the efficacy of such menin inhibitors (e.g., Compound I). As such, there is a pressing need for adding a cellular efflux transporter inhibitor, such as a P-gp inhibitor or a BCRP inhibitor, to a treatment regimen using a menin inhibitor, e.g., for treating hematological malignancies, such as acute myeloid leukemia. The present disclosure addresses this need by providing methods of using a menin inhibitor and an inhibitor of a cellular efflux transporter, such as P-gp or BCRP, in situations such as treating hematological malignancies, such as acute leukemias or acute myeloid leukemia (AML), which will provide improved effects, such as improved efficacy (e.g., therapeutic efficacy or cellular efficacy), increased drug permeability into cells (e.g., increased cellular concentration of the menin inhibitor), increased cellular apoptosis, reduced cellular proliferation or metastasis, increased bioavailability for the menin inhibitor, or increased distribution of the menin inhibitor in tissues. The methods and compositions provided herein may be useful for treating diseases dependent on the activity of menin, MLL1, and/or MLL2, such as hematological malignancies.

In certain aspects, the present disclosure provides a method for treating a hematological malignancy in a subject in need thereof comprising administering to the subject a menin inhibitor and a cellular efflux transporter inhibitor, in particular a P-gp inhibitor. Alternatively, the cellular efflux transporter inhibitor is a BCRP inhibitor.

In certain aspects, the present disclosure provides a method for treating a hematological malignancy in a subject in need thereof who is being treated with a cellular efflux transporter inhibitor, in particular a P-gp inhibitor, comprising administering to the subject a menin inhibitor. Alternatively, the cellular efflux transporter inhibitor is a BCRP inhibitor.

In certain aspects, the present disclosure provides a method for increasing efficacy of a menin inhibitor for treating a hematological malignancy in a subject in need thereof, the method comprising administering to the subject a menin inhibitor and a cellular efflux transporter inhibitor, particularly a P-gp inhibitor. Alternatively, the cellular efflux transporter inhibitor is a BCRP inhibitor.

In certain aspects, the present disclosure provides a method for increasing permeability of a menin inhibitor into cells (e.g., cancer cells) in a subject comprising administering to the subject a menin inhibitor and a cellular efflux transporter inhibitor, particularly a P-gp inhibitor. Alternatively, the cellular efflux transporter inhibitor is a BCRP inhibitor.

In certain aspects, the present disclosure provides a method of increasing distribution of a menin inhibitor in tissues (e.g., cancer tissues or brain tissues) in a subject comprising administering to the subject a menin inhibitor and a cellular efflux transporter inhibitor, particularly a P-gp inhibitor. Alternatively, the cellular efflux transporter inhibitor is a BCRP inhibitor.

In certain aspects, the present disclosure provides a method for increasing bioavailability of a menin inhibitor in a subject comprising administering to the subject a menin inhibitor and a cellular efflux transporter inhibitor, particularly a P-gp inhibitor. Alternatively, the cellular efflux transporter inhibitor is a BCRP inhibitor.

In some embodiments, the menin inhibitor is Compound I:

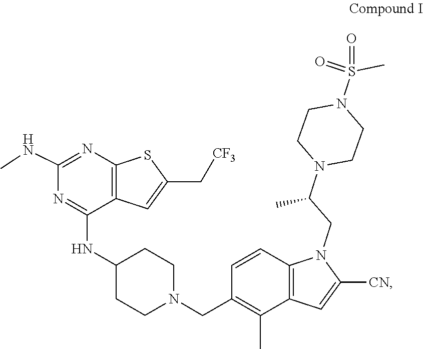

or a stereoisomer, tautomer, or isotopolog thereof, or a prodrug any of the foregoing, or a pharmaceutically acceptable salt of any of the foregoing, or a solvate of any of the foregoing (collectively, pharmaceutically acceptable forms thereof).

In certain aspects, the present disclosure also provides a kit comprising (1) a therapeutically effective amount of a pharmaceutical composition comprising a menin inhibitor and a pharmaceutically acceptable carrier or excipient, in a first dosage form; and (2) a therapeutically effective amount of a pharmaceutical composition comprising a P-gp inhibitor and a pharmaceutically acceptable carrier or excipient, in a second dosage form.

In certain aspects, the present disclosure also provides a kit comprising (1) a therapeutically effective amount of a pharmaceutical composition comprising a menin inhibitor and a pharmaceutically acceptable carrier or excipient, in a first dosage form; and (2) a therapeutically effective amount of a pharmaceutical composition comprising a BCRP inhibitor and a pharmaceutically acceptable carrier or excipient, in a second dosage form.

In yet another aspect, the present disclosure provides a compound that is 4-methyl-5-((4-((2-(methylamino)-6-((S)-2,2,2-trifluoro-1-hydroxyethyl)thieno[2,3-d]pyrimidin-4-yl)amino)piperidin-1-yl)methyl)-1-((S)-2-(4-(methylsulfonyl)piperazin-1-yl)propyl)-1H-indole-2-carbonitrile (Compound IB) having the structure of:

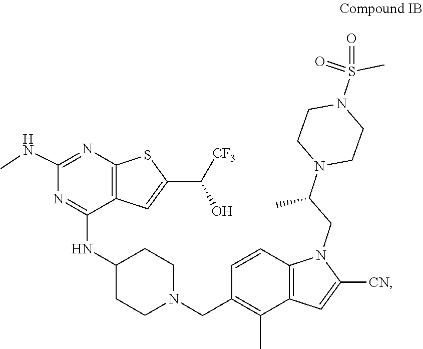

or a pharmaceutically acceptable form thereof. In yet another aspect, the present disclosure provides a pharmaceutical composition comprising Compound IB or a pharmaceutically acceptable form thereof, and a pharmaceutically acceptable excipient or carrier. In a further aspect, the present disclosure provides methods for treating a hematological malignancy in a subject in need thereof, the method comprising administering to the subject Compound IB or a pharmaceutically acceptable form thereof.

In yet another aspect, the present disclosure provides a compound that is 4-methyl-5-((4-((2-(methylamino)-6-((R)-2,2,2-trifluoro-1-hydroxyethyl)thieno[2,3-d]pyrimidin-4-yl)amino)piperidin-1-yl)methyl)-1-((S)-2-(4-(methylsulfonyl)piperazin-1-yl)propyl)-1H-indole-2-carbonitrile (Compound IC) having the structure of:

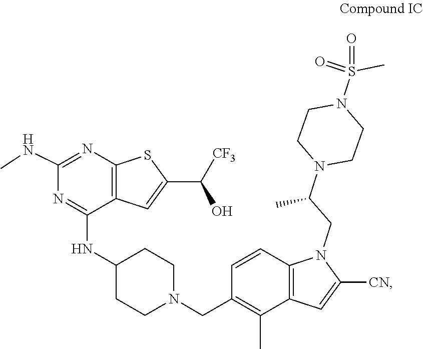

or a pharmaceutically acceptable form thereof. In yet another aspect, the present disclosure provides a pharmaceutical composition comprising Compound IC or a pharmaceutically acceptable form thereof, and a pharmaceutically acceptable excipient or carrier. In a further aspect, the present disclosure provides methods for treating a hematological malignancy in a subject in need thereof, the method comprising administering to the subject Compound IC or a pharmaceutically acceptable form thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the MDR1-MDCK (Madin Darby Canine Kidney) monolayer assay as an in vitro model for the identification and characterization of P-gp substrates and inhibitors.

FIGS. 2A-2B show efflux ratios for Compound I, Compound IA, Compound IB, SNDX-5613, VTP-50469, Compound A, and digoxin (reference) in the MDCK monolayer assay when tested alone, in the presence of a P-gp inhibitor/BCRP inhibitor (elacridar (GF120918), 10 μM), or in the presence of posaconazole (2 μM or 4 μM). FIG. 2A: efflux ratios for Compound I, Compound IA, and Compound IB; FIG. 2B: efflux ratios for Compound I, SNDX-5613, VTP-50469, and Compound A.

FIG. 3 shows the development of a P-gp-expressing KMT2A(MLL)-rearranged cell line model (MV4;11) by lentiviral transduction and selection for the P-gp open reading frame (ORF) (MV4;11-PGP). FIG. 3 also shows the expression levels of P-gp mRNA in the MV4;11 parental cell line, MV4;11-Control cell line, and MV4;11-PGP cell line that were measured by qPCR (WT level set to 1).

FIG. 4 illustrates measured luminescence curves of the MV4;11 parental, MV4;11-Control, and MV4;11-PGP cell lines, which were treated with Compound I only, to determine the IC₅₀ values of Compound I against such treated cell lines.

FIGS. 5A-5B illustrate measured luminescence curves of the MV4;11-Control and MV4;11-PGP cell lines, which were treated with Compound I only (DMSO), Compound I+posaconazole (1.5 μM; FIG. 5A), and valspodar (1 μM; FIG. 5B), respectively, to determine the IC₅₀ values of Compound I against such treated cell lines.

FIGS. 6A-6J illustrate cell viability curves of the MV4; 11-Control and MV4;11-PGP cell lines when exposed to test compound alone or in combination with posaconasole (Posa) or valspodar (Val) as described in Example 4. FIG. 6A: Compound I with or without posaconazole; FIG. 6B: Compound I with or without valspodar; FIG. 6C: SNDX-5613 with or without posaconazole; FIG. 6D: SNDX-5613 with or without valspodar; FIG. 6E: Compound A with or without posaconazole; FIG. 6F: Compound A with or without valspodar; FIG. 6G: Compound B with or without posaconazole; FIG. 6H: Compound B with or without valspodar; FIG. 6I: Compound C with or without posaconazole; FIG. 6J: Compound C with or without valspodar.

FIGS. 7A-7D illustrate cell viability curves of the MOLM13-Control and MOLM13-PGP cell lines, which were treated with Compound I or SNDX-5613 with or without posaconazole (Posa) or valspodar (Val) as described in Example 4. FIG. 7A: Compound I with or without posaconazole; FIG. 7B: Compound I with or without valspodar; FIG. 7C: SNDX-5613 with or without posaconazole; FIG. 7D: SNDX-5613 with or without valspodar.

FIGS. 8A-8D illustrate the cell viability curves of the OCI-AML3-Control and OCI-AML3-PGP cell lines, which were treated with Compound I or SNDX-5613, with or without posaconazole (Posa) or valspodar (Val) as described in Example 4. FIG. 8A: Compound I with or without posaconazole; FIG. 8B: Compound I with or without valspodar; FIG. 8C: SNDX-5613 with or without posaconazole; FIG. 8D: SNDX-5613 with or without valspodar.

### DETAILED DESCRIPTION OF THE INVENTION

The present disclosure provides compositions and methods useful for treating proliferative diseases (e.g., cancers such as hematological malignancies). In some embodiments, the methods use menin inhibitors and a second agent. In some embodiments, the second agent is a cellular efflux transporter inhibitor, particularly a P-gp inhibitor or a breast cancer resistance protein (BCRP) inhibitor.

In one aspect, provided herein are therapies using a menin inhibitor and a cellular efflux transporter inhibitor, particularly a P-gp inhibitor or a breast cancer resistance protein (BCRP) inhibitor.

Results provided herein demonstrate that certain menin inhibitors (e.g., Compound I) are substrates of P-gp and that administration of a menin inhibitor (e.g., Compound I) with a P-gp inhibitor results in increased menin inhibitor exposure (i.e., menin inhibitor concentration) in cells as compared to administration of a menin inhibitor alone. As shown in the Examples section below, Compound I was shown to be a substrate of P-gp using a MDR1-MDCK monolayer assay, and IC₅₀ values for menin inhibitors increased in P-gp-expressing cells but recovered when the cells were treated with the menin inhibitor and a P-gp inhibitor.

### Definitions

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain. The terms “C_{x-y} alkenyl” and “C_{x-y} alkynyl” refer to substituted or unsubstituted straight-chain or branched-chain unsaturated hydrocarbon groups that contain at least one double or triple bond respectively. Unless stated otherwise specifically in the specification, a C_{x-y} alkyl, C_{x-y} alkenyl, or C_{x-y} alkynyl is optionally substituted by one or more substituents such as those substituents described herein.

“Carbocycle” refers to a saturated, unsaturated, or aromatic ring in which each atom of the ring is a carbon atom. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

“Heterocycle” refers to a saturated, unsaturated, or aromatic ring comprising one or more heteroatoms. Exemplary heteroatoms include N, O, Si, P, B, and S atoms, and preferably N, O, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryls as defined above which are optionally substituted by one or more substituents such as those substituents described herein.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, and aromatic and non-aromatic substituents of organic compounds. The permissible substituents can be one or more and the same or different for appropriate organic compounds. For purposes of this disclosure, heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic, and a heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂—R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—X(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Where substituent groups are specified by their conventional chemical formulae, written from left to right, they equally encompass the chemically identical substituents that would result from writing the structure from right to left, e.g., —CH₂O— is equivalent to —OCH₂—.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Enriching for deuterium may afford certain therapeutic advantages, such as increased in vivo half-life and/or exposure, or may provide a compound useful for investigating in vivo routes of drug elimination and metabolism. Isotopically enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E-, and tautomeric forms as well.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

As used herein, “efficacy” or “effective” refers to cellular efficacy, in vivo efficacy, therapeutic efficacy (such as anticancer efficacy or antileukemic efficacy), or prophylactic efficacy, as appropriate for the context. In the cellular context, efficacy can be measured to rate of apoptosis, extent of cellular proliferation, gene expression, protein levels, IC₅₀ values, or enzymatic percent inhibition. Increased efficacy may include one or more effects selected from increasing apoptosis, decreasing cellular proliferation, decreasing IC₅₀, and increasing enzymatic percent inhibition. In an in vivo context, such as in animal models, efficacy can be measured by duration of survival or tumor volume. Increased efficacy may therefore include increased survival, a decrease in the rate of tumor volume increase, or a decrease in tumor volume. In the clinical or antileukemic context, efficacy can be measured by rates of complete response (CR; can be with or without measurable residual disease, MRD+/−), complete response with partial hematologic recovery (CRh), CR/CRh, complete response with incomplete hematologic recovery (CRi), complete remission with incomplete platelet recovery (CRp), composite complete remission (CRC=CR+CRi (incl. CRp)+CRh), overall response rate (ORR; ORR=CR (MRD+ or MRD−)+CRh+morphologic leukemic-free state (MLFS)), partial response (PR), stable disease without progression (SD), overall survival (OS), blast count levels, duration of remission (DOR), time to relapse, event-free survival, progression-free survival, and other measures. Increase efficacy means that one or more such measures is improved (e.g., increased CR/CRh rate, increased OS, reduced blast count levels, increased ANC).

A “therapeutic effect,” as that term is used herein, encompasses a therapeutic benefit and/or a prophylactic benefit as described above. A prophylactic effect includes delaying or eliminating the appearance or re-appearance of a disease or condition, delaying or eliminating the onset of symptoms of a disease or condition, or slowing, halting, or reversing the progression of a disease or condition, or any combination thereof.

The terms “administered with” or “administered in combination with,” and their grammatical equivalents, as used herein, encompass administration of two or more agents to an animal, including a human, so that both agents and/or their metabolites are present in the subject at the same time. Such administration includes simultaneous administration in separate compositions, administration at different times in separate compositions, or administration in a composition in which both agents are present.

In some embodiments, the menin inhibitor and the P-gp inhibitor or BCRP inhibitor may be administered “concurrently,” meaning with overlapping dosing schedules, e.g., at the same time or approximately the same time, or on the same day, or “sequentially,” meaning one after the other in non-overlapping dosing schedules. In some embodiments, administration of the agents may be initiated at different times and then continue to be administered concurrently (e.g., administration of the menin inhibitor followed by administration of the menin inhibitor and the P-gp or BCRP inhibitor, or the reverse). In some embodiments, the menin inhibitor continues to be administered while the P-gp inhibitor or BCRP inhibitor administration is stopped or is paused for a period of time.

The term “expression” refers to the process by which a polynucleotide is transcribed into mRNA and/or the process by which the transcribed mRNA (also referred to as a “transcript”) is subsequently translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell. The level of expression (or alternatively, the “expression level”) of a HOXA9 gene can be determined, for example, by determining the level of HOXA9 polynucleotides, polypeptides, and/or gene products. “Differentially expressed” or “differential expression” as applied to a nucleotide sequence (e.g., a gene) or polypeptide sequence in a subject, refers to the differential production of the mRNA transcribed and/or translated from the nucleotide sequence or the protein product encoded by the nucleotide sequence. A differentially expressed sequence may be overexpressed or under-expressed as compared to the expression level of a reference sample (i.e., a reference level). As used herein, elevated expression levels or overexpression refer to an increase in expression, generally at least 1.25-fold, or alternatively, at least 1.5-fold, or alternatively, at least 2-fold, or alternatively, at least 3-fold, or alternatively, at least 4-fold, or alternatively, at least 10-fold expression over that detected in a reference sample. As used herein, underexpression is a reduction in expression and generally is at least 1.25-fold, or alternatively, at least 1.5-fold, or alternatively, at least 2-fold, or alternatively, at least 3-fold, or alternatively, at least 4-fold, or alternatively, at least 10-fold expression under that detected in a reference sample. Underexpression also encompasses absence of expression of a particular sequence as evidenced by the absence of detectable expression in a test subject when compared to a reference sample. In some embodiments, a subject to be treated with a menin inhibitor expresses P-gp or BCRP. In some embodiments, that expression is at a level that is above a reference level.

“Subject” refers to an animal, such as a mammal, for example a human. The methods described herein can be useful in both human therapeutics and veterinary applications. In some embodiments, the subject is a mammal, and in some embodiments, the subject is human. “Mammal” includes humans and both domestic animals such as laboratory animals (e.g., rats, mice) and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compound of Formula (I-A), Formula (I-B), Formula (II-A), Formula (III-A), Formula (IV-A), or Formula (VI-B), or Compound I, IA, IB, IC, A, B, or C). Thus, the term “prodrug” refers to a precursor of a biologically active compound that is pharmaceutically acceptable. In some aspects, a prodrug is inactive when administered to a subject but is converted in vivo to an active compound, for example, by hydrolysis. The prodrug compound often offers advantages of solubility, tissue compatibility, or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press) each of which is incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of prodrugs include, but are not limited to, acetate, formate, and benzoate derivatives of a hydroxy functional group, or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

“Pharmaceutically acceptable carrier or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The terms “antagonist” and “inhibitor” are used interchangeably, and they refer to a compound having the ability to inhibit a biological function (e.g., activity, expression, binding, protein-protein interaction) of a target protein (e.g., menin, MLL1, MLL2, and/or a KMT2A(MLL) fusion protein). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “menin inhibitor” refers to a compound that binds to the menin protein and/or inhibits the protein-protein interaction of menin with a KMT2A(MLL) protein (e.g., MLL1, MLL2, or KMT2A(MLL) fusion protein). In some embodiments, such binding or inhibition is selective. In certain embodiments, the menin inhibitor modulates the menin protein by binding to or interacting with one or more amino acids and/or one or more metal ions. Certain menin inhibitors may occupy the F9 and/or P13 pocket of menin. The binding of a menin inhibitor may disrupt menin or KMT2A(MLL) (e.g., MLL1, MLL2, or a KMT2A(MLL) fusion protein) downstream signaling. In certain embodiments, a menin inhibitor covalently binds menin and inhibits the interaction of menin with MLL. In certain embodiments, a menin inhibitor interacts non-covalently with menin and inhibits the interaction of menin with MLL. In some embodiments, a menin inhibitor has an IC₅₀ of less than 1 μM, or less than 500 nM, or less than 250 nM, or less than 100 nM, in a cellular assay in a cell line with MLL(KMT2A) fusion, such as MV4;11 or in a biochemical assay for MLL(4-43)/menin binding.

The present disclosure provides compounds for modulating the interaction of menin with proteins such as MLL1, MLL2, and KMT2A(MLL) fusion oncoproteins. In certain embodiments, the disclosure provides compounds and methods for inhibiting the interaction of menin with its upstream or downstream signaling molecules including, but not limited to, MLL1, MLL2, and KMT2A(MLL) fusion oncoproteins. Compounds of the disclosure may be used in methods for the treatment of a variety of cancers and other diseases associated with one or more of MLL1, MLL2, KMT2A(MLL) fusion proteins, and menin, such as hematological malignancies.

### Menin Inhibitors

In some embodiments, the menin inhibitor is a compound of Formula (I-A):

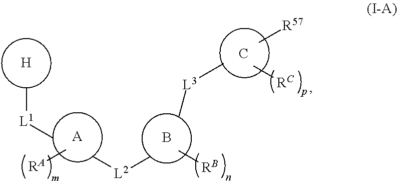

or a pharmaceutically acceptable form thereof, wherein:
H is selected from C₅₋₁₂ carbocycle and 5- to 12-membered heterocycle, each of which is optionally substituted with one or more R⁵⁰;
A is selected from bond, C₃₋₁₂ carbocycle and 3- to 12-membered heterocycle;
B is selected from C₃₋₁₂ carbocycle and 3- to 12-membered heterocycle;
C is 3- to 12-membered heterocycle;
L¹, L², and L³ are each independently selected from bond, —O—, —S—, —N(R⁵¹)—, —N(R⁵¹)CH₂—, —C(O)—, —C(O)O—, —OC(O)—, —OC(O)O—, —C(O)N(R⁵¹)—, —C(O)N(R⁵¹)C(O)—, —C(O)N(R⁵¹)C(O)N(R⁵¹)—, —N(R⁵¹)C(O)—, —N(R⁵¹)C(O)N(R⁵¹)—, —N(R⁵¹)C(O)O—, —OC(O)N(R⁵¹)—, —C(NR⁵¹)—, —N(R⁵¹)C(NR⁵¹)—, —C(NR⁵¹)N(R⁵¹)—, —N(R⁵¹)C(NR⁵¹)N(R⁵¹)—, —S(O)₂—, —OS(O)—, —S(O)O—, —S(O)—, —OS(O)₂—, —S(O)₂O—, —N(R⁵¹)S(O)₂—, —S(O)₂N(R⁵¹)—, —N(R⁵¹)S(O)—, —S(O)N(R⁵¹)—, —N(R⁵¹)S(O)₂N(R⁵¹)—, and —N(R⁵¹)S(O)N(R⁵¹)—; and alkylene, alkenylene, alkynylene, heteroalkylene, heteroalkenylene, and heteroalkynylene, each of which is optionally substituted with one or more R⁵¹, wherein two R⁵⁰ groups attached to the same atom or different atoms of any one of L, L², or L can together optionally form a bridge or ring;
R^{A}, R^{B}, and R^{c} are each independently selected at each occurrence from R⁵⁰, or two R^{A} groups, two R^{B} groups, or two R^{C} groups attached to the same atom or different atoms can together optionally form a bridge or ring;
m, n, and p are each independently an integer from 0 to 6;
R⁵⁰ is independently selected at each occurrence from:
   halogen, —NO₂, —CN, —OR, —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR²C(O)NR³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), and —P(O)(NR⁵²)₂; or two R⁵⁰ groups attached to the same atom taken together form ═O, ═S, or ═N(R⁵²);
   C₁₋₁₀ alkyl, C₂₋₁₀ alkenyl, and C₂₋₁₀ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R¹², —S(═O)₂R⁵², —S(═O)₂N(R⁵²)₂, —S(═O)₂NR⁵³R⁵⁴, —NR⁵²S(═O)₂R⁵², —NR¹²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R¹²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle, wherein each C₃₋₁₂ carbocycle and 3- to 12-membered heterocycle in R⁵⁰ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR¹²S(═O)₂NR⁵³R¹⁴, —C(O)R⁵², —C(O)OR¹², —OC(O)R¹², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₁₋₆ alkyl, Ct-6 haloalkyl, C₂₋₆ alkenyl, and C₂₋₆ alkynyl;
R⁵¹ is independently selected at each occurrence from:
   hydrogen, —C(O)R⁵², —C(O)OR⁵², —C(O)N(R⁵²)₂, —C(O)NR⁵³R⁵⁴;
   C₁₋₆ alkyl, C₂₋₆ alkenyl, and C₂₋₆ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R¹²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₂ carbocycle and 3- to 12-membered heterocycle in R⁵¹ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl;
R⁵² is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;
R⁵³ and R⁵⁴ are taken together with the nitrogen atom to which they are attached to form a heterocycle; and
R⁵⁷ is selected from:
   halogen, —NO₂, —CN, —SR, —NR⁵³R⁵⁴, S(═O)R², —S(═O)₂R², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)OR⁵², —OC(O)R¹², —OC(O)OR¹², —OC(O)N(R⁵²)₂, —OC(O)NR⁵³R⁵⁴, —NR⁵²C(O)OR⁵², —NR⁵²C(O)N(R⁵²)₂, —NR⁵²C(O)NR⁵³R⁵⁴, —C(O)NH(C₁₋₆ alkyl), —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═S, ═N(R⁵²); and
   C₁₋₁₀ alkyl, C₂₋₁₀ alkenyl, and C₂₋₁₀ alkynyl, each of which is substituted at each occurrence with one or more substituents independently selected from —NO₂, —CN, —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —P(O)(OR⁵²)₂, —P(O)(R¹²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═S, and =N(R⁵²);
wherein when C is azetidinylene, piperidinylene or piperazinylene and R⁵⁷ is —S(═O)₂R⁵⁸, —S(═O)₂N(R⁵²)₂, or —NR⁵²S(═O)₂R⁵²:
   p is an integer from 1 to 6; and/or
   L³ is substituted with one or more R⁵⁰, wherein L³ is not —CH₂CH(OH)—.

In some embodiments, the menin inhibitor is a compound of Formula (I-B):

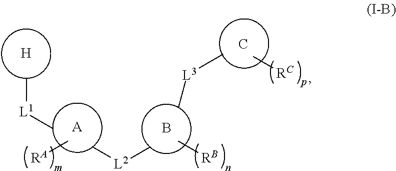

or a pharmaceutically acceptable form thereof, wherein:
H is selected from C₅₋₁₂ carbocycle and 5- to 12-membered heterocycle, each of which is optionally substituted with one or more R⁵⁰
A, B, and C are each independently selected from C₃₋₁₂ carbocycle and 3- to 12-membered heterocycle;
L¹ and L² are each independently selected from bond, —O—, —S—, —N(R⁵¹)—, —N(R⁵¹)CH₂—, —C(O)—, —C(O)O—, —OC(O)—, —OC(O)O—, —C(O)N(R⁵¹)—, —C(O)N(R⁵¹)C(O)—, —C(O)N(R⁵¹)C(O)N(R⁵¹)—, —N(R⁵¹)C(O)—, —N(R⁵¹)C(O)N(R⁵¹)—, —N(R⁵¹)C(O)O—, —OC(O)N(R⁵¹)—, —C(NR⁵¹)—, —N(R⁵¹)C(NR⁵¹)—, —C(NR⁵¹)N(R⁵¹)—, —N(R⁵¹)C(NR⁵¹)N(R⁵¹)—, —S(O)₂—, —OS(O)—, —S(O)O—, —S(O)—, —OS(O)₂—, —S(O)₂O—, —N(R⁵¹)S(O)₂—, —S(O)₂N(R⁵¹)—, —N(R⁵¹)S(O)—, —S(O)N(R⁵¹)—, —N(R⁵¹)S(O)₂N(R⁵¹)—, and —N(R⁵¹)S(O)N(R⁵¹)—; and alkylene, alkenylene, alkynylene, heteroalkylene, heteroalkenylene, and heteroalkynylene, each of which is optionally substituted with one or more R⁵⁰;
L³ is selected from alkylene, alkenylene, and alkynylene, each of which is substituted with one or more R⁵⁶ and optionally further substituted with one or more R⁵⁰;
R^{A}, R^{B}, and R^{C} are each independently selected at each occurrence from R⁵⁰; or two R^{A} groups, two R^{B} groups, or two R^{C} groups attached to the same atom or different atoms can together optionally form a bridge or ring;
m, n, and p are each independently an integer from 0 to 6;
R⁵⁰ is independently selected at each occurrence from:
   halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), and —P(O)(NR⁵²)₂, or two R⁵⁰ groups attached to the same atom taken together form ═O, ═S, or =N(R⁵²);
   C₁₋₁₀ alkyl, C₂₋₁₀ alkenyl, and C₂₋₁₀ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₂ carbocycle and 3- to 12-membered heterocycle in R⁵⁰ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R¹²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl;
R⁵¹ is independently selected at each occurrence from:
   hydrogen, —C(O)R⁵², —C(O)OR⁵², —C(O)N(R⁵²)₂, —C(O)NR⁵³R⁵⁴;
   C₁₋₆ alkyl, C₂₋₆ alkenyl, and C₂₋₆ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R¹²)₂, —NR⁵²S(═O)₂NR⁵³R¹⁴, —C(O)R¹², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R¹²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₂ carbocycle and 3- to 12-membered heterocycle in R⁵¹ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl;
R⁵² is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;
R⁵³ and R⁵⁴ are taken together with the nitrogen atom to which they are attached to form a heterocycle;
R⁵⁶ is independently selected at each occurrence from:
   —NO₂, —OR⁵⁹, —SR⁵², —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R¹²)₂, —C(O)NR³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, C₁₋₁₀ alkyl, C₂₋₁₀ alkenyl, C₂₋₁₀ alkynyl, C₃₋₁₂ carbocycle, and 3- to 12-membered heterocycle, or two R⁵⁶ groups attached to the same atom taken together form ═O, ═S, or =N(R⁵²);
   wherein each C₁₋₁₀ alkyl, C₂₋₁₀ alkenyl, and C₂₋₁₀ alkynyl in R⁵⁶ is optionally substituted at each occurrence with one or more substituents independently selected from halogen, —NO₂, —CN, —OR⁵⁹, —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R¹²)₂, —NR⁵²S(═O)₂NR⁵³R¹⁴, —C(O)R¹², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R¹²)₂, —P(O)(OR⁵²)(R⁵²), —P(O)(NR⁵²)(R⁵²), —NR⁵²P(o)(R⁵²), —P(O)(NR⁵²)(OR⁵²), —P(O)(NR⁵²)₂, ═O, ═S, ═N(R⁵²), C₃₋₁₂ carbocycle, and 3- to 12-membered heterocycle;
   wherein each C₃₋₂ carbocycle and 3- to 12-membered heterocycle in R⁵⁶ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R¹²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R¹²)₂, —P(O)(OR⁵²)(R⁵²), —P(O)(NR⁵²)(R⁵²), —NR⁵²P(O)(R⁵²), —P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl; and
   further wherein R⁵⁶ optionally forms a bond to ring C; and
R⁵⁹ is independently selected at each occurrence from C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle,
wherein when R⁵⁶ is —CH₃, L³ is not further substituted with —OH, —NH₂, or —CN.

In some embodiments, for a compound of Formula (I-A) or (I-B), R^{C} is selected from —C(O)R⁵², —S(═O)R⁵², —S(═O)₂R⁵², —S(═O)₂N(R⁵²)₂, —S(═O)₂NR⁵³R⁵⁴, —NR⁵²S(═O)₂R⁵², =0, C₁₋₃ alkyl, and C₁₋₃ haloalkyl, or two R^{C} groups attached to different atoms can together form a C₁₋₃ bridge.

In some embodiments, the menin inhibitor is a compound of Formula (II-A):

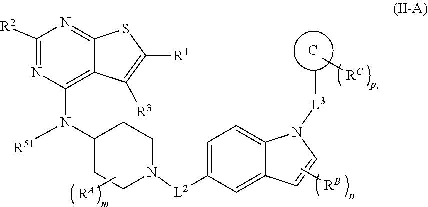

or a pharmaceutically acceptable form thereof, wherein:
C is selected from C₃₋₁₂ carbocycle and 3- to 12-membered heterocycle;
L² is selected from bond, —C(O)—, —C(O)O—, —C(O)N(R⁵¹)—, —C(O)N(R⁵¹)C(O)—, —C(O)N(R⁵¹)C(O)N(R⁵¹)—, —C(NR⁵¹)—, —S(O)₂—, —S(O)O—, —S(O)—, —S(O)₂O—, S(O)₂N(R⁵¹)—, and —S(O)N(R⁵¹)—; and alkylene, alkenylene, alkynylene, heteroalkylene, heteroalkenylene, and heteroalkynylene, each of which is optionally substituted with one or more R⁵⁰;
L³ is selected from alkylene, alkenylene, and alkynylene, each of which is substituted with one or more R⁵⁶ and optionally further substituted with one or more R⁵⁰;
R¹ and R³ are each independently selected from hydrogen and R⁵⁰;
R² is R⁵⁰;
R^{A}, R^{B}, and R^{C} are each independently selected at each occurrence from R⁵⁰, or two R^{A} groups, two R^{B} groups, or two R^{C} groups attached to the same atom or different atoms can together optionally form a bridge or ring;
m, n, and p are each independently an integer from 0 to 6;
R⁵⁰ is independently selected at each occurrence from:
   halogen, —NO₂, —CN, —OR⁵², —SR⁵², —N(R⁵²)₂, —N⁵³R⁵⁴, —S(═O)R⁵², —S(═O)₂R⁵², —S(═O)₂N(R⁵²)₂, —S(═O)₂NR⁵³R⁵⁴, —NR⁵²(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, and —P(O)(R⁵²)₂, or two R⁵⁰ groups attached to the same atom taken together form ═O, ═S, or =N(R⁵²);
   C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R¹², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₂ carbocycle and 3- to 12-membered heterocycle in R⁵⁰ is independently optionally substituted with one or more substituents selected from halogen, —NO₂, —CN, —OR⁵², —SR¹², —N(R¹²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R¹², —OC(O)OR⁵², —OC(O)N(R⁵²)₂, —OC(O)NR⁵³R⁵⁴, —NR⁵²C(O)R⁵², —NR⁵²C(O)OR¹², —NR¹²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R¹²)₂, ═O, ═S, ═N(R⁵²), C₁₋₆ alkyl, C₁₋₆ haloalkyl, C₂₋₆ alkenyl, and C₂₋₆ alkynyl;
R⁵¹ is independently selected at each occurrence from:
   hydrogen, —C(O)R⁵², —C(O)OR¹², —C(O)N(R⁵²)₂, and —C(O)NR⁵³R¹⁴; and
   C₁₋₆ alkyl, C₂₋₆ alkenyl, and C₂₋₆ alkynyl, each of which is independently optionally substituted at each occurrence with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R¹⁴, —C(O)R⁵², —C(O)OR⁵², —OC(O)R¹², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₂ carbocycle and 3- to 12-membered heterocycle in R⁵¹ is independently optionally substituted with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R¹⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₁₋₆ alkyl, C₁₋₆ haloalkyl, C₂₋₆ alkenyl, and C₂₋₆ alkynyl;
R⁵² is independently selected at each occurrence from hydrogen; and C₁₋₂₀ alkyl, C₂₋₂₀ alkenyl, C₂₋₂₀ alkynyl, C₁₋₆ heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;
R⁵³ and R⁵⁴ are taken together with the nitrogen atom to which they are attached to form a heterocycle, optionally substituted with one or more R⁵⁰;
R⁵⁶ is independently selected at each occurrence from: —NO₂, —OR⁵⁹, —SR⁵², —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, C₁-t alkyl, C₂₋₁₀ alkenyl, C₂₋₁₀ alkynyl, C₃₋₁₂ carbocycle, and 3- to 12-membered heterocycle, or two R⁵⁶ groups attached to the same atom taken together form ═O, ═S, or =N(R⁵²);
   wherein each C₁₋₁₀ alkyl, C₂₋₁₀ alkenyl, and C₂₋₁₀ alkynyl in R⁵⁶ is optionally substituted at each occurrence with one or more substituents independently selected from halogen, —NO₂, —CN, —OR⁵⁹, —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R¹², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle;
   wherein each C₃₋₂ carbocycle and 3- to 12-membered heterocycle in R⁵⁶ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₁₋₆ alkyl, C₁₋₆ haloalkyl, C₂₋₆ alkenyl, and C₂₋₆ alkynyl; and
   further wherein R⁵⁶ optionally forms a bond to ring C; and
R⁵⁹ is independently selected at each occurrence from C₁₋₂₀ alkyl, C₂₋₂₀ alkenyl, C₂₋₂₀ alkynyl, C₁₋₆ heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₂ carbocycle, or 3- to 6-membered heterocycle,
wherein when R⁵⁶ is —CH₃, L³ is not further substituted with —OH, —NH₂, or —CN.

In some embodiments, the menin inhibitor is a compound of Formula (III-A):

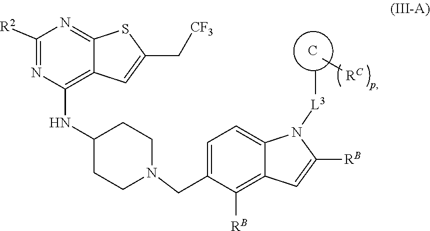

or a pharmaceutically acceptable form thereof, wherein R², R^{B}, R^{C}, L³, C, and p are each defined as described for Formula (II-A).

In some embodiments, the menin inhibitor is a compound of Formula (IV-A) or Formula (IV-B):

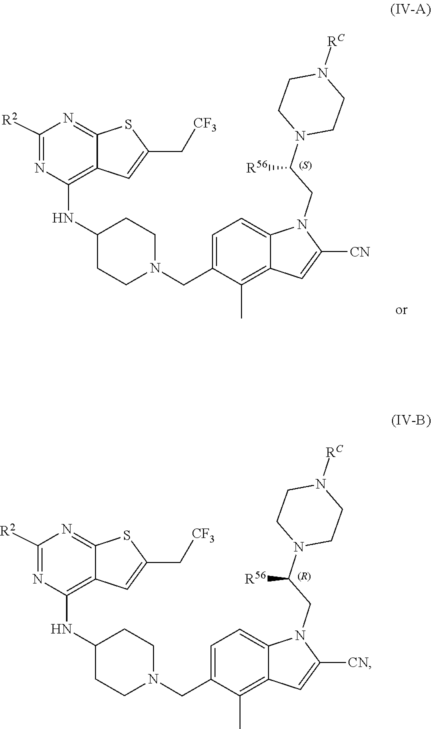

or a pharmaceutically acceptable form thereof, wherein R², R⁵⁶, and R^{C} are each defined as described for Formula (II-A).

In some embodiments, the menin inhibitor is Compound I:

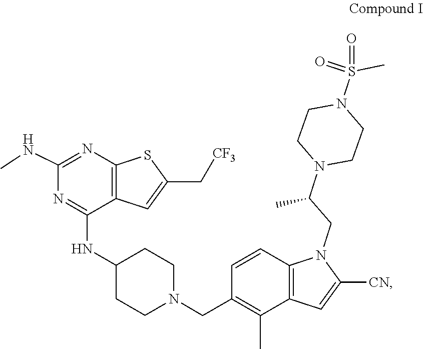

or a pharmaceutically acceptable form thereof, such as a pharmaceutically acceptable salt or solvate thereof.

In some embodiments, the menin inhibitor is Compound IA:

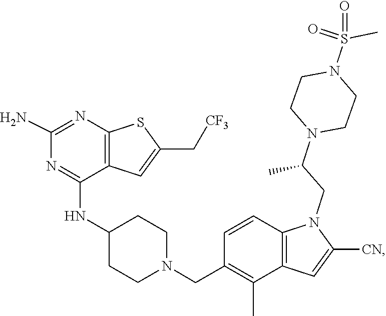 [SMILES: Cc1c(CN2CCC(Nc3nc(N)nc4sc(CC(F)(F)F)cc34)CC2)ccc2c1cc(C#N)n2C[C@H](C)N1CCN(S(C)(=O)=O)CC1]

or a pharmaceutically acceptable form thereof.

In some embodiments, the menin inhibitor is Compound IB:

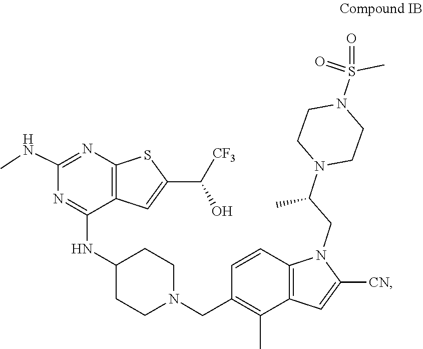

or a pharmaceutically acceptable form thereof.

In some embodiments, the menin inhibitor is a menin inhibitor described in U.S. Pat. No. 10,683,302, which disclosure is incorporated by reference herein. In some embodiments, the menin inhibitor is a compound of Formula (A-I):

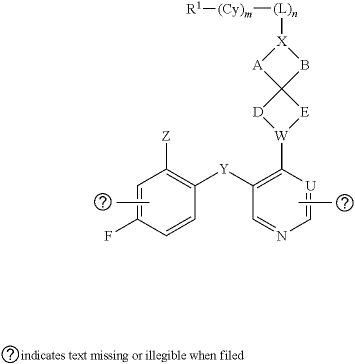

or a pharmaceutically acceptable form thereof, wherein:
A, B, D, and E are each independently selected from —C(R^{A1})(R^{A2}), —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A2}—, —C(R^{A1})(R^{A2})—C(═O)—, —C(═O)—, or —N═C(NH₂)—;
U is N or CR^{U}, wherein R^{U} is H, halo, CN, OH, C₁₋₄ alkyl, C₁₋₄ alkoxy, amino, C₁₋₄ alkyl amino, or C₂₋₈ dialkylamino;
W is N or CR^{W}, wherein R^{W} is H, halo, CN, OH, C₁₋₄ alkyl, C₁₋₄ alkoxy, amino, C₁₋₄ alkyl amino, or C₂₋₈ dialkylamino;
X is N or CR^{X}, wherein R^{X} is H, halo, CN, OH, C₁₋₄ alkyl, C₁₋₄ alkoxy, amino, C₁₋₄ alkyl amino, or C₂₋₈ dialkylamino, wherein when X is N, the atom of L that is directly bonded with X is other than N, O, or S;
L is selected from —C₁₋₆ alkylene- and —(C₁₋₄ alkylene)ₐ-Q-(C₁₋₄ alkylene)_{b}-, wherein the C₁₋₆ alkylene group and any C₁₋₄ alkylene group of the —(C₁₋₄ alkylene)ₐ-Q-(C₁₋₄ alkylene)_{b}— group is optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino;
Q is —O—, —S—, —S(═O)—, —S(═O)₂—, —C(═O)—, —C(═O)NR^{q1}—, —C(═O)—, —OC(═O)NR^{q1}—, —NR^{q1}—, —NR^{q1}C(═O)O—, —NR^{q1}C(═O)NR^{q1}—, —S(═O)₂NR^{q1}—, —C(═NR^{q1})—, or —C(═NR^{q2})—NR^{q1}—, wherein each R^{q1} is independently selected from H or C₁₋₆alkyl, and wherein each R^{q2} is independently selected from H, C₁₋₆ alkyl, and CN;
Cy is a linking C₆₋₁₄ aryl, C₃₋₁₈ cycloalkyl, 5-16 membered heteroaryl, or 4-18 membered heterocycle group, each of which is optionally substituted with 1, 2, 3, or 4 substituents independently selected from R^{Cy}, wherein the heteroaryl or heterocycle has 1-3 rings and 1-4 heteroatoms independently selected from nitrogen, sulfur, and oxygen;
each R^{Cy} is independently selected from halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycle, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycle are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the heteroaryl or heterocycle has 1-3 rings and 1-4 heteroatoms independently selected from nitrogen, sulfur, and oxygen,
R¹ is H, Cy¹, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2} and S(O)₂NR^{c2}R^{d2}, wherein said C₁₋₆alkyl, C₂₋₆ alkenyl, and C₂₋₆ alkynyl are each optionally substituted by 1, 2, 3, or 4 substituents independently selected from halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
Y is O, S, CR^{Y1}R^{Y2} or NR^{Y3}, wherein R^{Y1}, R^{Y2}, and R^{Y3} are each independently selected from 11 and C₁₋₄ alkyl;
Z is Cy², halo, C₁₋₆ alkyl, C₁₋₄ haloalkyl, C₁₋₄ cyanoalkyl, C₂₋₆ alkenyl, C₂₋₆ alkynyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and P(O)R^{c3}R^{d3} wherein said C₁₋₆ alkyl, C₂₋₆ alkenyl, and C₂₋₆ alkynyl are each optionally substituted by 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{e3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R² and R³ is independently selected from H, halo, C₁₋₆ alkyl, C₁₋₄ haloalkyl, C₁₋₄ cyanoalkyl, C₂₋₆ alkenyl, C₂₋₆alkynyl, CN, NO₂, OR^{a4}, SR^{a4}, C(O)R^{b1}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆alkenyl, and C₂₋₆ alkynyl are each optionally substituted by 1, 2, 3, or 4 substituents independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR⁴C(═NR^{e4})NR^{c4}R^{d4}, NR^{c4}R^{d4}, NR⁴C(O)R^{b4}, NR⁴C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
each R^{A1} is independently selected from H, halo, C₁₋₄ alkyl, C₁₋₄ alkoxy, C₁₋₄ haloalkyl, C₁₋₄ haloalkoxy, amino, C₁₋₄ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₄ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR^{z}, wherein said C₁₋₄ alkyl is optionally substituted by phenyl, C₁₋₄ alkoxy, C₁₋₄ haloalkoxy, CN, NO₂, or OH;
R^{z} is H, C₁₋₄ alkyl, or phenyl;
each Cy¹ is independently selected from C₆₋₁₄ aryl, C₃₋₁₈ cycloalkyl, 5-16 membered heteroaryl, and 4-18 membered heterocycle, each of which is optionally substituted with 1, 2, 3, or 4 substituents independently selected from R^{Cy1}, wherein the heteroaryl or heterocycle has 1-3 rings and 1-4 heteroatoms independently selected from nitrogen, sulfur, and oxygen;
each Cy² is independently selected from C₆₋₁₄ aryl, C₃₋₁₈ cycloalkyl, 5-16 membered heteroaryl, and 4-18 membered heterocycle, each of which is optionally substituted with 1, 2, 3, or 4 substituents independently selected from R^{Cy2}, wherein the heteroaryl or has 1-3 rings and 1-4 heteroatoms independently selected from nitrogen, sulfur, and oxygen;
each R^{Cy1} and R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycle, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, phenyl, C₃₋₇ cycloalkyl, 5-6 membered heteroaryl, and 4-7 membered heterocycle are each optionally substituted by 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5} NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein the heteroaryl or heterocycle has 1-3 rings and 1-4 heteroatoms independently selected from nitrogen, sulfur, and oxygen;
each R^{a1}, R^{b1}, R^{c1}, R^{d1}, R^{a2}, R^{b2}, R^{c2}, R^{d2}, R^{a3}, R^{b3}, R^{c3}, R^{d3}, R^{a4}, R^{b4}, R^{c4}, R^{d4}, R^{a5}, R^{b5}, R^{c5}, and R^{d5} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycle, C₆₋₁₀ aryl-C₁₋₆ alkyl, C₃₋₁₀ cycloalkyl-C₁₋₆ alkyl, (5-10 membered heteroaryl)-C₁₋₆ alkyl, and (4-10 membered heterocycle)-C₁₋₆ alkyl, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycle, C₆₋₁₀ aryl-C₁₋₆ alkyl, C₃₋₁₀ cycloalkyl-C₁₋₆ alkyl, (5-10 membered heteroaryl)-C₁₋₆ alkyl, and (4-10 membered heterocycle)-C₁₋₆ alkyl are each optionally substituted with 1, 2, 3, 4, or 5 substituents independently selected from R^{g}, wherein the heteroaryl or heterocycle has 1-3 rings and 1-4 heteroatoms independently selected from nitrogen, sulfur, and oxygen,
each R^{e1}, R^{e2}, R^{e3}, R^{e4}, and R^{e5} is independently selected from H, C₁₋₄ alkyl, and CN;
each R^{g} is independently selected from the group consisting of OH, NO₂, CN, halo, C₁₋₆ alkyl, C₂₋₆alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
n is 0 or 1;
m is 0 or 1;
p is 0, 1, 2, or 3;
q is 0, 1, or 2;
a is 0 or 1; and
b is 0 or 1,
wherein any cycloalkyl or heterocycle group is optionally further substituted by 1 or 2 oxo groups.

In one embodiment, the menin inhibitor is SNDX-5613, which has the structure of:

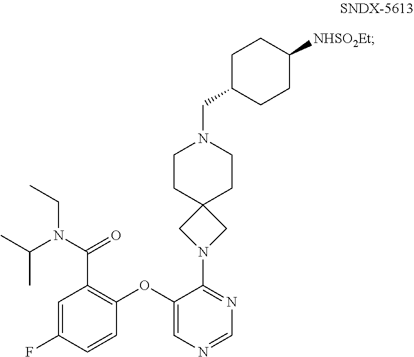

or a pharmaceutically acceptable form thereof.

In another embodiment, the menin inhibitor is VTP-50469, which has the structure of:

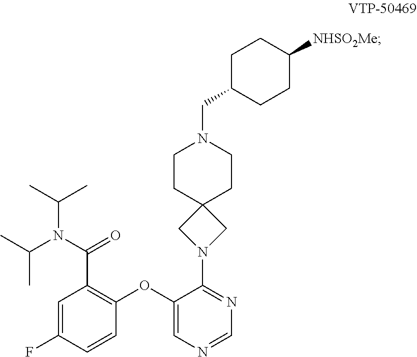

or a pharmaceutically acceptable form thereof.

In some embodiments, the menin inhibitor is a menin inhibitor described in U.S. Pat. Publ. No. 20210269454, which disclosure is incorporated by reference herein. In some embodiments, the menin inhibitor is a compound of Formula (A-II):

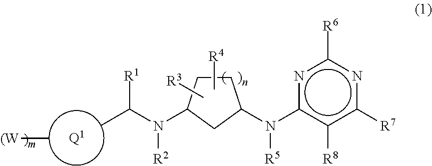

wherein
the dotted circle indicates that the ring is aromatic, R¹ and R² are each independently a hydrogen atom or a C₁₋₆ alkyl group, one of R³ and R⁴ is a hydrogen atom, a hydroxy group, a halogen atom, a C₁₋₆ alkoxy group, a di(C₁₋₆ alkyl)carbamoyl group, or an oxazolyl group, and the other of R³ and R⁴ is a hydrogen atom, a hydroxy group, a halogen atom, or a C₁₋₆ alkoxy group,
R⁵ is a hydrogen atom, a C₁₋₆ alkyl group, or a hydroxy C₁₋₆ alkyl group,
R⁶ is a hydrogen atom, a C₁₋₆ alkyl group, a halogen atom, a C₁₋₆ alkoxy group, an amino group, or a C₁₋₆ alkylamino group.
R⁷ and R⁸ are taken together with the carbon atom to which R⁷ is bonded and the carbon atom to which R⁸ is bonded to form any of the following formulas (2A) to (2C):

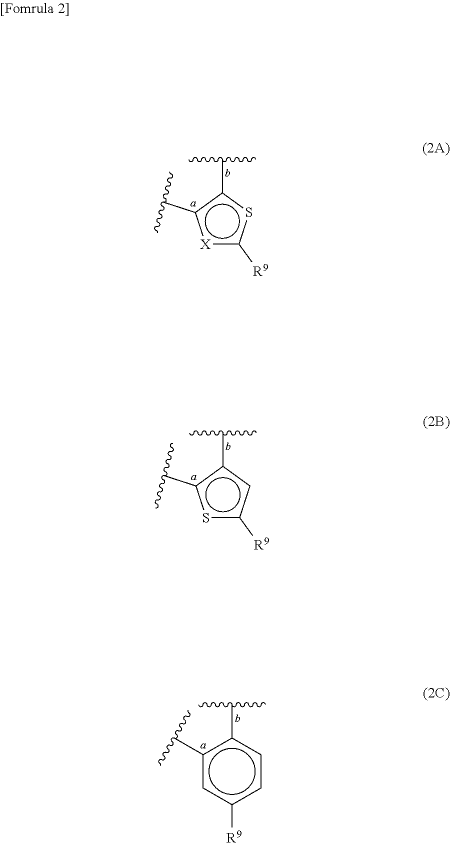

wherein
the dotted circle indicates that the ring is aromatic,
the carbon atom marked with a is the carbon atom to which R⁸ is bonded,
the carbon atom marked with b is the carbon atom to which R⁷ is bonded,
X is CH or a nitrogen atom, and
R⁹ is a halogen, C₁₋₆ alkyl group, a C₃₋₈ cycloalkyl group, a
C₃₋₈ cycloalkyl C₁₋₆ alkyl group, a C₁₋₆ alkoxy C₁₋₆ alkyl group, or an oxetanyl group, or R⁷ is a hydrogen atom, and R⁸ is the following formula (3).

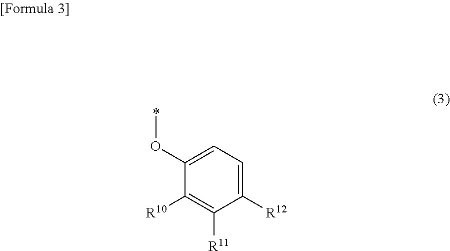

wherein
* indicates a bonding site,
R¹⁰ is a di(C₁₋₆ alkyl) carbamoyl group, a (C₁₋₆ alkyl)pyrimidinyl group, a (C₁₋₆ alkyl)phenyl group, or a (C₁₋₆ alkyl)pyrazolyl group,
R¹¹ is a hydrogen atom or a halogen atom, and
R¹² is a halogen atom,
m is 1 or 0,
n is 1 or 2,
Ring Q¹ is a 6-membered aromatic ring optionally containing one nitrogen atom in the ring (the aromatic ring optionally has one or two substituents independently selected from the following Group A), a 5-membered aromatic heterocycle containing, in the ring, one or two heteroatoms independently selected from the group consisting of a nitrogen atom and a sulfur atom (the aromatic heterocycle optionally has one substituent independently selected from the following Group A), a C₃₋₈ cycloalkane ring optionally having one substituent independently selected from the following Group A, a C₄₋₈ cycloalkene ring optionally having one substituent independently selected from the following Group A, a 4- to 8-membered saturated heterocycle containing one nitrogen atom in the ring (the saturated heterocycle optionally has one substituent independently selected from the following Group A), or a 9-membered bicyclic aromatic heterocycle containing one nitrogen atom in the ring (the bicyclic aromatic heterocycle optionally has one or two substituents independently selected from the following Group B), and
W is the following formula (4A) or (4B):

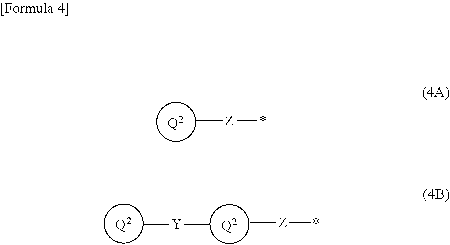

wherein
* indicates a bonding site,
Ring Q² is a 6-membered aromatic ring optionally containing one nitrogen atom in the ring (the aromatic ring optionally has one to three substituents independently selected from the following Group C), a 6-membered aromatic heterocycle containing two nitrogen atoms in the ring (the aromatic heterocycle optionally has one to three substituents independently selected from the following Group C), a 5-membered aromatic heterocycle containing, in the ring, one to three heteroatoms independently selected from the group consisting of a nitrogen atom, an oxygen atom and a sulfur atom (the aromatic heterocycle optionally has one substituent independently selected from the following Group C), a 9- or 10-membered bicyclic aromatic or partially unsaturated heterocycle containing, in the ring, one to three heteroatoms independently selected from the group consisting of a nitrogen atom and an oxygen atom (the bicyclic aromatic or partially unsaturated heterocycle optionally has one or two substituents independently selected from the following Group D), a 5- to 8-membered saturated heterocycle containing, in the ring, one or two heteroatoms independently selected from the group consisting of an oxygen atom and a nitrogen atom (the saturated heterocycle optionally has one substituent independently selected from the following Group E), or a C₃₋₈ cycloalkane ring optionally having one substituent independently selected from the following Group E, Ring Q³ is a 4- to 8-membered saturated heterocycle containing one nitrogen atom or one oxygen atom in the ring (the saturated heterocycle optionally has one C₁₋₆ alkylsulfonyl group), or a 6-membered aromatic ring optionally containing one nitrogen atom in the ring (the aromatic ring optionally has one substituent independently selected from the following Group F),
Y is a single bond or an oxygen atom, and
Z is a single bond, an oxygen atom, —NH—, —SO₂—, a C₁₋₆ alkylene group, *—R¹³—NHC(═O)—**, *—R⁴—O—**, or *—R⁵—NH—**,
wherein * is bonded to Ring Q², ** is bonded to Ring Q¹, and R¹³, R¹⁴ and R¹⁵ are each independently a C₁₋₆ alkylene group,
   Group A: a halogen atom, a hydroxy group, a C₁₋₆ alkyl group, a C₁₋₆ alkoxy group, a hydroxy C₁₋₆ alkoxy group, a vinylsulfonylamino(C₁₋₆ alkyl)carbamoyl group, and a prop-2-enoylamino(C₁₋₆ alkyl)carbamoyl group,
   Group B: a cyano group, a C₁₋₆ alkyl group, a halogen atom, and a C₁₋₆ alkoxy group,
   Group C: a halogen atom, a C₁₋₆ alkyl group, a C₁₋₆ alkoxy group, a C₁₋₆ alkyl(C₁₋₆ alkylsulfonyl)amino group, a cyano group, a C₁₋₆ alkylsulfonyl group, a C₁₋₆ alkylamino group, a di(C₁₋₆ alkyl)amino group, a halogeno C₁₋₆ alkyl group, a C₁₋₆ alkoxy C₁₋₆ alkoxy group, a halogeno C₁₋₆ alkoxy group, a C₁₋₆ alkylsulfonyl C₁₋₆ alkyl group, a di(C₁₋₆ alkyl)sulfamoyl group, a C₁₋₆ alkylenedioxy group, a (C₁₋₆ alkyl)carbamoyl group, a hydroxy C₁₋₆ alkyl group, a 2-C₃₋₅ alkenoylamino group, a C₁₋₆ alkyl (2-C₃₋₆alkenoyl)amino group, a hydroxy group, an oxo group, a —OC(²H)₃ group, and a —N[(C(²H)₃]₂ group,
   Group D: a halogen atom, a C₁₋₆ alkyl group, and a C₁₋₆ alkylsulfonyl group,
   Group E: an oxo group, a hydroxy group, and a C₁₋₆ alkoxy group, and
   Group F: a halogen atom, and a C₁₋₆ alkoxy group.

In one embodiment, the menin inhibitor is Compound A, which has the structure of:

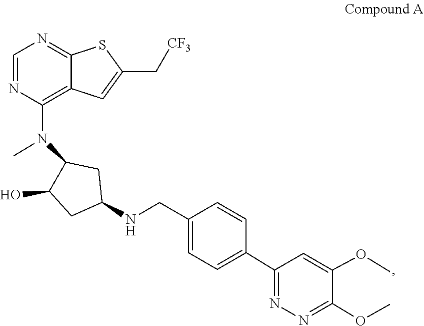 [SMILES: COc1cc(-c2ccc(CN[C@H]3C[C@@H](O)[C@@H](N(C)c4ncnc5sc(CC(F)(F)F)cc45)C3)cc2)nnc1OC]

or a pharmaceutically acceptable form thereof.

In some embodiments, the menin inhibitor is a menin inhibitor described in PCT Publ. No. WO2021/121327, which disclosure is incorporated by reference herein. In some embodiments, the menin inhibitor is a compound of Formula (A-III):

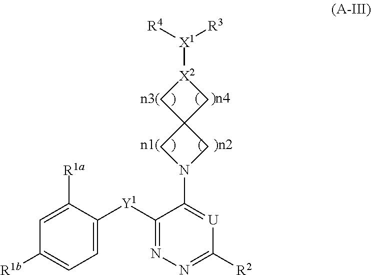

or a pharmaceutically acceptable form thereof, wherein
R^{1a} represents —C(═O)—NR^{xa}R^{xb}; Het; or

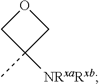

Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c}—;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}, C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl;
wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR¹²R^{12b}); and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;
R^{9a}, R^{9b}, R^{10a}, R^{10b}, R^{10c}, R¹¹, R^{12a}, and R^{12b} are each independently selected from the group consisting of hydrogen and C₁₋₆alkyl.

In one embodiment, the menin inhibitor is Compound B, which has the structure of:

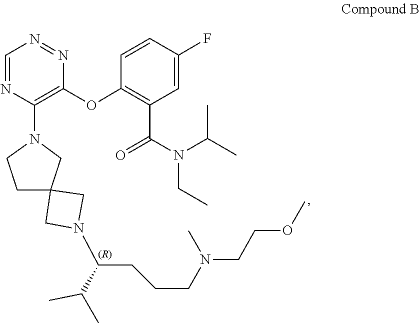

or a pharmaceutically acceptable form thereof.

In some embodiments, the menin inhibitor is a menin inhibitor described in U.S. Pat. No. 11,084,825, which disclosure is incorporated by reference herein. In some embodiments, the menin inhibitor is a compound of Formula (A-IV):

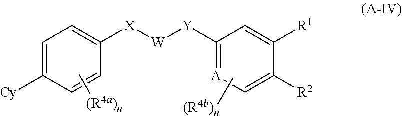

or a pharmaceutically acceptable form thereof,
wherein:
A is N;
Cy is:

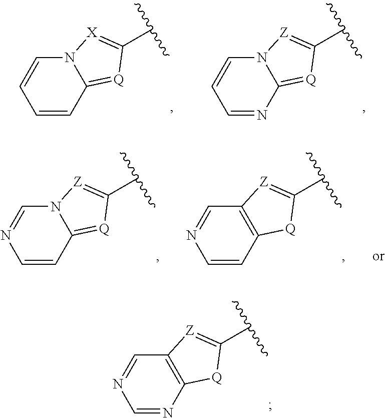

wherein:
Q is ═N—, —NH—, —O—, or —S—; and
Z is —CR^{5a}═ or —N═;
wherein Cy is optionally substituted with one or more independently selected R⁷ substituents;
X is —C(R^{3b})₂—, —NR³a-, or —O—;
W is —C(O)—, —S(O)—, or —S(O)₂—;
Y is a single bond, —C(R^{3b})₂—, —NR^{3a}—, or —O—;
   (0) (i) R¹ is H, halo, CN, C₁₋₆ alkyl, or C₁₋₆ haloalkyl; and
R² is CH₂-Cy²-NHC(O)—C(R^{6a})═C(R^{6b})(R^{6c}) or Cy²-NHC(O)—C(R^{6a})═C(R^{6b})(R^{6c}); or
(ii) R¹ is CH₂—Cy²-NHC(O)—C(R^{6a})═C(R^{6b})(R^{6c}) or Cy²-NHC(O)—C(R^{6a})═C(R^{6b})(R^{6c}); and
R² is H, halo, CN, C₁₋₆ alkyl, or C₁₋₆ haloalkyl;
each R^{3a} is independently H or C₁₋₆ alkyl;
each R^{3b} is independently H or C₁₋₆ alkyl;
each R^{4a} is independently H, halo, CN, C₁₋₆ alkyl, C(O)R, C(O)N(R)₂, C(O)OR, N(R)₂, NRC(O)R, OR, S(O)₂R, C₃₋₇ cycloalkyl, a 4- to 7-membered heterocycloalkyl ring, phenyl, an 8- to 10-membered bicyclic aryl ring, or a 5- or 6-membered heteroaryl ring, wherein the 4- to 7-membered heterocycloalkyl ring has 1 or 2 heteroatoms independently selected from the group consisting of nitrogen, oxygen, and sulfur, and the 5- or 6-membered heteroaryl ring has 1, 2, 3, or 4 heteroatoms independently selected from the group consisting of nitrogen, oxygen, and sulfur,
each R^{4b} is independently H, halo, CN, C₁₋₆ alkyl, C(O)R, C(O)N(R)₂, C(O)OR, N(R)₂, NRC(O)R, OR, S(O)₂R, C₃₋₇ cycloalkyl, a 4- to 7-membered heterocycloalkyl ring, phenyl, an 8- to 10-membered bicyclic aryl ring, or a 5- or 6-membered heteroaryl ring, wherein the 4- to 7-membered heterocycloalkyl ring has 1 or 2 heteroatoms independently selected from the group consisting of nitrogen, oxygen, and sulfur, and the 5- or 6-membered heteroaryl ring has 1, 2, 3, or 4 heteroatoms independently selected from the group consisting of nitrogen, oxygen, and sulfur;
each R⁷ is independently a 4- to 7-membered heterocycloalkyl ring, phenyl, an 8- to 10-membered bicyclic aryl ring, or a 5- or 6-membered heteroaryl ring, wherein each 4- to 7-membered heterocycloalkyl ring independently has 1 or 2 heteroatoms independently selected from the group consisting of nitrogen, oxygen, and sulfur, and each 5- or 6-membered heteroaryl ring independently has 1, 2, 3, or 4 heteroatoms independently selected from the group consisting of nitrogen, oxygen, and sulfur, and further wherein each 4- to 7-membered heterocycloalkyl ring, phenyl, 8- to 10-membered bicyclic aryl ring, and 5- or 6-membered heteroaryl ring is optionally and independently substituted with one or more substituents independently selected from the group consisting of halo, CN, C₁₋₆ alkyl, C₁₋₆ haloalkyl, NH₂, NH(C₁₋₆ alkyl), N(C₁₋₆ alkyl)₂, OH, and O(C₁₋₆ alkyl);
each R is independently H, C₁₋₆ aliphatic, a saturated or partially unsaturated 4- to 7-membered heterocyclic ring, phenyl, an 8- to 10-membered bicyclic aryl ring, or a 5- or 6-membered heteroaryl ring, wherein the saturated or partially unsaturated 4- to 7-membered heterocyclic ring has 1 or 2 heteroatoms independently selected from the group consisting of nitrogen, oxygen, and sulfur, and the 5- or 6-membered heteroaryl ring has 1, 2, 3, or 4 heteroatoms independently selected from the group consisting of nitrogen, oxygen, and sulfur; or
two geminal R groups, together with the nitrogen atom to which they are attached, form a saturated or partially unsaturated 4- to 7-membered heterocyclic ring or a 5- or 6-membered heteroaryl ring, wherein the 4- to 7-membered heterocyclic ring or the 5- or 6-membered heteroaryl ring has 0, 1, 2, or 3 additional heteroatoms independently selected from the group consisting of nitrogen, oxygen, and sulfur;
R^{5a} is H, halo, CN, C₁₋₆ alkyl, or C₁₋₆ haloalkyl;
R^{6a} is H or C₁₋₆ alkyl;
R^{6b} is H or C₁₋₆ alkyl; or
R^{6a} and R^{6b}, joined together, form a single bond;
R^{6c} is H or C₁₋₆ alkyl, wherein the C₁₋₆ alkyl is optionally substituted with N(CH₃)₂;
Cy² is a 4- to 7-membered heterocycloalkyl ring, phenyl, or pyridyl, wherein the 4- to 7-membered heterocycloalkyl ring has 1 or 2 heteroatoms independently selected from the group consisting of nitrogen, oxygen, and sulfur;
m is 1, 2, or 3, and
n is 1, 2, 3, or 4.

In one embodiment, the menin inhibitor is Compound C, which has the structure of:

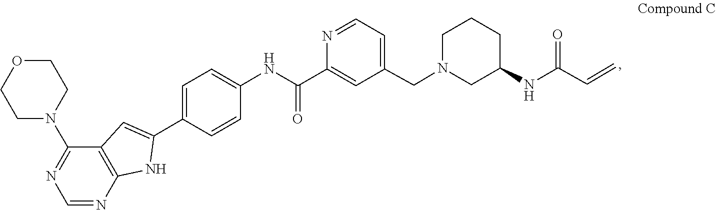 [SMILES: C=CC(=O)N[C@@H]1CCCN(Cc2ccnc(C(=O)Nc3ccc(-c4cc5c(N6CCOCC6)ncnc5[nH]4)cc3)c2)C1]

or a pharmaceutically acceptable form thereof.

In some embodiments, the menin inhibitor is Compound I, Compound IA, Compound IB, Compound IC, SNDX-5613, VTP-50469, Compound A, Compound B, or Compound C, or a pharmaceutically acceptable form thereof.

In some embodiments, the menin inhibitor is Compound I, SNDX-5613, VTP-50469, Compound A, Compound B, or Compound C, or a pharmaceutically acceptable form thereof.

In some embodiments, the menin inhibitor is Compound I, Compound IA, Compound IB, Compound IC, SNDX-5613, VTP-50469, or Compound A, or a pharmaceutically acceptable form thereof.

In some embodiments, the menin inhibitor is Compound I, SNDX-5613, VTP-50469, or Compound A, or a pharmaceutically acceptable form thereof.

In some embodiments, the menin inhibitor is Compound I, Compound IA, or Compound IB, or a pharmaceutically acceptable form thereof.

In some embodiments, the menin inhibitor is Compound IA or Compound IB, or a pharmaceutically acceptable form thereof. In some embodiments, the menin inhibitor is Compound IC, or a pharmaceutically acceptable form thereof.

The compound of Formula (I-A), Formula (I-B), Formula (II-A), Formula (III-A), Formula (IV-A), or Formula (VI-B) (e.g., Compound I) may be synthesized by methods described in U.S. Pat. No. 10,781,218, which disclosure is incorporated by reference herein.

In yet another aspect, the present disclosure also provides a compound of 4-methyl-5-((4-((2-(methylamino)-6-((S)-2,2,2-trifluoro-1-hydroxyethyl)thieno[2,3-d]pyrimidin-4-yl)amino)piperidin-1-yl)methyl)-1-((S)-2-(4-(methylsulfonyl)piperazin-1-yl)propyl)-1H-indole-2-carbonitrile (Compound IB) having the structure of:

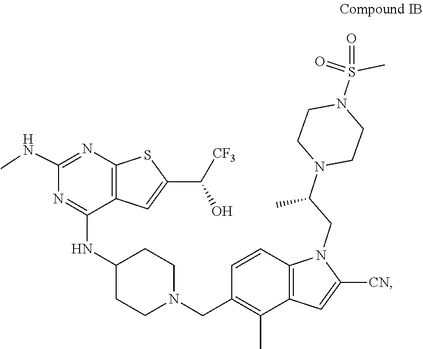

or a pharmaceutically acceptable form thereof (e.g., a salt, stereoisomer, tautomer, prodrug, solvate, or isotopolog thereof). In yet another aspect, the present disclosure provides a pharmaceutical composition comprising Compound IB or a pharmaceutically acceptable form thereof (salt, stereoisomer, tautomer, prodrug, solvate, or isotopolog thereof), and a pharmaceutically acceptable excipient or carrier (e.g., a pharmaceutically acceptable excipient or carrier described herein). In a further aspect, the present disclosure provides methods for treating a hematological malignancy in a subject in need thereof, the method comprising administering to the subject a therapeutically effective amount of Compound IB or a pharmaceutically acceptable form thereof (e.g, a salt, stereoisomer, tautomer, prodrug, solvate, or isotopolog thereof).

In yet another aspect, the present disclosure provides a compound that is 4-methyl-5-((4-((2Iethylamino)-6-((R)-2,2,2-trifluoro-1-hydroxyethyl)thieno[2,3-d]pyrimidin-4-yl)amino)piperidin-1-yl)methyl)-1-((S)-2-(4-(methylsulfonyl)piperazin-1-yl)propyl)-1H-indole-2-carbonitrile (Compound IC) having the structure of:

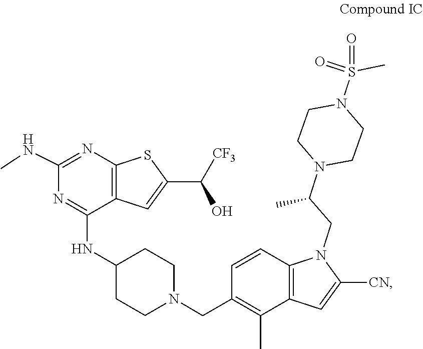

or a pharmaceutically acceptable form thereof. In yet another aspect, the present disclosure provides a pharmaceutical composition comprising Compound IC or a pharmaceutically acceptable form thereof, and a pharmaceutically acceptable excipient or carrier. In a further aspect, the present disclosure provides methods for treating a hematological malignancy in a subject in need thereof, the method comprising administering to the subject Compound IC or a pharmaceutically acceptable form thereof.

In some embodiments, the hematological malignancy is selected from the group consisting of: Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute lymphocytic leukemia (ALL), mixed phenotype acute leukemia (MPAL), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma (CML), and chronic monocytic leukemia.

In some embodiments, the hematological malignancy is AML. In some embodiments, the hematological malignancy is MM. In some embodiments, the hematological malignancy is CLL. In some embodiments, the hematological malignancy is DLBCL. In some embodiments, the hematological malignancy is MIDS. In some embodiments, the hematological malignancy is CML. In some embodiments, the hematological malignancy is relapsed or refractory. In some embodiments, the hematological malignancy is relapsed or refractory AML.

### P-Glycoprotein Inhibitors

Cellular efflux transporters include P-gp inhibitors. There are literature reports of various in vitro and in vivo models used for assessing P-gp interactions (see, e.g., Leahey E., et al. JAMA 1978, 240:533-534; Lin J., Yamazaki M., Clin. Pharmacokinet. 2003; 42:59-98; Matheny C. et al., Pharmacotherapy 2001, 21:778-796). In vitro assays that measure aspects such as ATPase activity, rhoadmine-123 uptake, calcein AM uptake, cell-based bidirectional permeability, and radioligand binding, along with in vivo models such as transgenic (knockout mice) animal models are used in discovery settings. A cell-based bidirectional permeability assay using digoxin as a probe has been used for determining the P-gp inhibition potential of test compounds in drug discovery laboratories (see, e.g., Balimane P. V. et al., AAPS J. 2006, 8:E1-13; Keogh J. P. et al., Eur. J. Pharm. Sci. 2006, 27:543-554; Rautio J. et al., Drug Metab. Dispos. 2006, 34:786-792). The FDA guidance document (see, “In Vitro Drug Interaction Studies—Cytochrome P450 Enzyme- and Transporter-Mediated Drug Interactions, Guidance for Industry,” U.S. Dept. of Health and Human Services, FDA (CDER), January 2020, available at: https://www.fda.gov/media/134582/download) also recommends conducting bidirectional permeability studies using Caco-2 cells or other cell lines (e.g., MDCK, LLC-PK₁, etc.), either wild-type or transfected (with P-gp), to determine the P-gp inhibition potential of test compounds (see, e.g., Pastan et al., Proc. Natl. Acad. Sci. 1988, 85:4486-4490).

In some embodiments, the “percent inhibition of P-gp” as used herein refers to the inhibition potential or capability of a compound that has an inhibitory effect on P-gp. The percent inhibition of P-gp can be determined using methods known to those skilled in the art. In some embodiments, the P-gp inhibitors described herein have a percent inhibition of P-gp of greater than 10%, 20%, 30%, 40%, 50%, 60%, 70%, 80%, or 90%. In some embodiments, the P-gp inhibitor has a percent inhibition of P-gp of greater than 20%, 30%, 40%, 50%, or 60%.

In certain embodiments, the percent inhibition of P-gp is measured based on apical to basolateral (A to B) transport as well as basolateral to apical (B to A) transport of [³H]-digoxin through a Caco-2 cell monolayer or a monolayer of another suitable cell line (e.g., MDCK, LLC-PK₁, etc.), measured in the absence and presence of the tested P-gp inhibitor. In some embodiments, the measurements are performed using MDCK monolayers.

In some embodiments, the P-gp inhibitor reduces efflux ratio of the menin inhibitor by at least 20%, 30%, 40%, 50%, 60%, 70%, 80%, or 90% relative to the efflux ratio of the menin inhibitor in the absence of the P-gp inhibitor.

In certain embodiments, the change in efflux ratio can be calculated using the difference of the efflux ratio of the P-gp substrate in the presence of the P-gp inhibitor as compared to in the absence of the P-gp inhibitor.

The apparent permeability coefficient Pₐₚₚ (cm/s) was calculated using the equation:

$${P}_{app} = ( {dC}_{r} / dt ) \times {V}_{r} / ( A \times {C}_{0} )$$

where dCᵣ/dt is the cumulative concentration of compound in the receiver chamber as a function of time (μM/s); Vᵣ is the solution volume in the receiver chamber (0.075 mL on the apical side, 0.25 mL on the basolateral side); A is the surface area for the transport, i.e. 0.0804 cm² for the area of the monolayer; C₀ is the initial concentration in the donor chamber (μM).

The efflux ratio can be calculated using the equation:

$$Efflux Ratio = {P}_{app} ( BA ) / {P}_{app} ( AB )$$

Percent recovery can be calculated using the equation:

$$\% Recovery = 100 \times [ ( {V}_{r} \times {C}_{r} ) + ( {V}_{d} \times {C}_{d} ) ] / ( {V}_{d} \times {C}_{0} )$$

where V_{d} is the volume in the donor chambers (0.075 mL on the apical side, 0.25 mL on the basolateral side); C_{d} and Cᵣ are the final concentrations of transport compound in donor and receiver chambers, respectively.

In some embodiments, the P-gp inhibitor increases the Pₐₚₚ (AB) of the P-gp substrate (e.g., the menin inhibitor) and/or decreases the Pₐₚₚ (BA) of the P-gp substrate (e.g., the menin inhibitor). In one embodiment, the P-gp inhibitor increases the Pₐₚₚ (AB) of the P-gp substrate (e.g., the menin inhibitor). In one embodiment, the P-gp inhibitor decreases the Pₐₚₚ (BA) of the P-gp substrate (e.g., the menin inhibitor).

In some embodiments, the inhibitory potential of a P-gp inhibitor can be determined by measuring the percent inhibition of P-gp by the P-gp inhibitor. In some embodiments, the inhibitory potential of a P-gp inhibitor can be determined by measuring the changes in potency of the menin inhibitor in cancer cells that express P-gp (cancer cells transfected with P-gp). In some embodiments, the P-gp inhibitor decreases the IC₅₀ of the menin inhibitor in inhibiting proliferation of cancer cells that express P-gp (cancer cells transfected with P-gp) by at least 20%, 30%, 40%, 50%, 60%, 70%, 80%, or 90% relative to the IC₅₀ of the menin inhibitor alone.

In certain embodiments, the percent inhibition of P-gp can be calculated using the calculation Method 1 below:

$$Percent inhibition of P - gp = [ ( BAi - ABi ) / ( Bac - ABc ) ] \times 100$$

where, BAc and ABc are the B to A and A to B permeability of digoxin alone, BAi and ABi are the B to A and A to B permeability of digoxin in the presence of the test compound (P-gp inhibitor).

In certain embodiments, the percent inhibition of P-gp can be calculated using the calculation Method 2 below:

$$Percent inhibition of P - gp = [ ( ERc - ERi ) / ( ERc - 1 ) ] \times 100$$

where, ERc is the efflux ratio of digoxin alone and ERi was the efflux ratio of digoxin in the presence of the test compound (P-gp inhibitor), and Efflux Ratio (ER) is the ratio of B to A/A to B permeability.

In certain embodiments, the percent inhibition of P-gp can be calculated using the calculation Method 3 below:

$$Percent inhibition of P - gp = [ ( BAc - BAi ) / ( BAc - 50 nm / s ) ] \times 100$$

where, BAc is the B to A permeability value in nanometers per second of digoxin alone, and BAi is the B to A permeability value in nanometers per second of digoxin in the presence of the test compound (P-gp inhibitor). The correction factor of 50 nm/s used in the denominator is based on an experimental value lowest B to A permeability achievable (for digoxin in presence of a P-gp inhibitor).

In certain embodiments, the percent inhibition of P-gp can be calculated using the calculation Method 4 below:

$$Percent inhibition of P - gp = [ ( ABi - aBc ) / ( 50 nm / s - ABc ) ] \times 100$$

where, ABc is the A to B permeability value in nanometers per second of digoxin alone, and ABi is the A to B permeability value in nanometers per second of digoxin in the presence of the test compound (P-gp inhibitor). The correction factor of 50 nm/s used in the denominator is based on an experimental value highest A to B permeability achievable (for digoxin in presence of a P-gp inhibitor).

In some embodiments, the inhibitory potential of the P-gp inhibitors is measured by the reduction of the efflux ratio of the menin inhibitor. The efflux ratio of the menin inhibitor is the ratio of B to A/A to B permeability in through monolayer cells, e.g., Caco-2 cell or MDCK monolayers.

In some embodiments, the P-gp inhibitor is also an inhibitor of cytochrome P450 3A4 (Cyp3A4).

In some embodiments, the P-gp inhibitor is selected from the group consisting of amiodarone, aldosterone, azithromycin, capmatinib, clarithromycin, cobcistat, daclatasvir, diosmin, dronedarone, elagolix, elacridar, eliglustat, flibanserin, fostamatinib, glecaprevir and/or pibrentasvir, ivacaftor, lapatinib, ledipasvir, neratinib, osimeritinib, propafenone, ranolazine, rolapitant, simeprevir, tepotinib, tezacaftor and/or ivacaftor, ticagrelor, tucatinib, velpatasvir, vemurafenib, voclosporin, lidocaine, clomiphene, cefoperazone, cortisol, ceftriaxone, dexamethasone, erythromycin, prednisone, itraconazole, progesterone, chloroquine, emetine, desipramine, quinidine, trazodone, hydroxychloroquine, dipyridamole, quinacrine, reserpine, quinine, cyclosporin A, bepridil, colchicine, diltiazem, FK-506, felodipine, quercetin, SDZ PSC-833, nifedipine, SDZ 280-446, nisoldipine, terfenadine, nitrendipine, tumor necrosis factor, tiapamil, vitamin A, verapamil, etoposide, actinomycin D, daunorubicin, ketoconazole, mitomycin-C, tamoxifen, taxol, RU-486, trimetrexase, devapamil, vinblastine, gallopamil, vincristine, emopamil, indinavir, nelfinavir, saquinavir, ritonavir, phenothiazines, and bupivacaine.

In some embodiments, the P-gp inhibitor is an azole antifungal. In some embodiments, the P-gp inhibitor is selected from the group consisting of ketoconazole, itraconazole, hydroxyitraconazole, and posaconazole. In some embodiments, the P-gp inhibitor is a breast cancer resistance protein (BCRP) inhibitor.

### BCRP Inhibitors

In a further example of a cellular efflux transporter, breast cancer resistance protein (BCRP) is another ATP transport protein that contains a conserved ATP-binding cassette (see, e.g., Doyle et al., Proc. Natl. Acad. Sci. USA 95:15665-15670 (1998); Maliepaard et al., Cancer Res. 59:4559-4563 (1999). BCRP is the gene product of the Bcrp1/Mxr/Abcp/ABCG2 gene (Miyake et al., Cancer Res. 59:8-13 (1999))). The huBCRP cDNA was originally cloned from several different human tumor cell lines that were resistant to multiple drugs including doxorubicin, topotecan, and mitoxantrone. In contrast to the structure of the MDR1 gene, which consists of two duplicated halves, the predicted structure of BCRP is that of a “half transporter,” with a single ATP binding cassette and transmembrane region (see, e.g., Doyle et al. (1998)).

In some embodiments, the BCRP inhibitor is selected from the group consisting of oteseconazole, biochanin A, chrysin, curcumin, eltrombopag, gefitinib, imatinib, Ko143, KS-176, fumitremorgin C (FTC), elacridar (GF120918), YHO-13351, diethylstilbestrol, cyclosporine A, prazosin, saquinavir, ritonavir, β-estradiol, verapamil, tamoxifen, triclabendazole sulfoxide, Hoechst 33342, PCI 29732, ML753286, CP-100356 hydrochloride, ML230, FD 12-9, quercetin, novobiocin, WK-X-34, tryprostatin A, quizartinib, omeprazole, methotrexate, ergocristine, nicardipine, ethinylestradiol, astemizole, telodipine, glibenclamide, ketoconazole, chlorprotixene, nitrendipine, chlorpromazine, progesterone, mifepristone, dipyridamole, lopinavir, amlodarone, simvastatin, loperamide, terfenadine, clotrimazol, spironolactone, maprotiline, digoxin, quinine, fexofenadine, diltiazem, erythromycin, etoposide, prednisone, trimethoprim, chlorzoxazone, folic acid, lansoprazole, dexlansoprazole, rantidine, cimetidine, indomethacin, prednisolone, propanolol, timolol, desipramine, pravastatin, hydrocortisone, sulfinpyrazone, fenolibrate, tipranavir, nelfinavir, erlotinib, flupentixol, celecoxib, thioridazine, isradipine, fendiline, medroxyprogesterone, pramoxine, piroxicam, terazosin, diazoxide, oxazepam, propafenone, tinidazole, meclizine, tetracycline, budesonide, desmethyldiazepam, nevirapine, diazepam, zanamivir, flurbiprofen, neomycin sulfate, nitrofurantoin, valacyclovir, carbamazepine, chenodeoxychloic acid, hydrochlorothiazide, amantadine, amoxicillin, phenytoin, antipyrine, bendroflumethiazide, ganciclovir, metoclopramide, pindolol, warfarin, amiloride, bupivacaine, carisoprodol, nizatidine, orphenadrine, procyclidine, acyclovir, atropine, captopril, furosemide, hydralazine, levothyroxine, salicyclic acid, sotalol, valganciclovir, levodopa, methimazole, sulindac, metoprolol, zidovudine, gliclazide, mesalazine, bupropion, and sulfasalazine. In some embodiments, the BCRP inhibitor is β-estradiol, omeprazole, ethinylestradiol, ketoconazole, progesterone, simvastatin, spironolactone, prednisone, folic acid, lansoprazole, dexlansoprazole, pravastatin, hydrocortisone, or salicylic acid.

### Methods

The present disclosure provides a method for treating a proliferative disease (e.g., a cancer such as a hematological malignancy) in a subject in need thereof. The method may comprise administering to the subject a menin inhibitor such as those described herein (e.g., Compound I) and a cellular efflux transporter inhibitor, particularly (a) a P-gp inhibitor or (b) a BCRP inhibitor.

The present disclosure provides a method for treating a proliferative disease (e.g., a cancer such as a hematological malignancy) in a subject in need thereof who is being treated with a cellular efflux transporter inhibitor, particularly (a) a P-gp inhibitor or (b) a BCRP inhibitor. The method may comprise administering to the subject a menin inhibitor such as described herein (e.g., Compound I)).

The present disclosure provides a method for increasing efficacy of a menin inhibitor for treating a hematological malignancy in a subject in need thereof. The method may comprise administering to the subject a menin inhibitor (e.g., Compound I) and a cellular efflux transporter inhibitor, particularly (a) a P-gp inhibitor or (b) a BCRP inhibitor. In some embodiments, increasing efficacy comprises increasing therapeutic or antileukemic efficacy for a leukemia such as an acute leukemia. In some embodiments, increasing efficacy comprises improved measures of or is measured by one or more of CR (MRD+/−), CRh, CR/CRh, CRi, CRp, CRC, ORR, MLFS, PR, SD, OS, blast count levels, DOR, time to relapse, event-free survival, or progression-free survival, or ANC.

In yet another aspect, the present disclosure provides a method of increasing the efficacy of a menin inhibitor for inhibiting cancer cell proliferation (e.g., tumor growth, disease progression, or metastasis) comprising administering to the subject a menin inhibitor and a cellular efflux transporter inhibitor, particularly a P-gp inhibitor or a BCRP inhibitor. In yet another aspect, the present disclosure provides a method of increasing the clinical, therapeutic, or antileukemic efficacy of a menin inhibitor in a subject with a hematological malignancy comprising administering to the subject a menin inhibitor and a cellular efflux transporter inhibitor, particularly a P-gp inhibitor or a BCRP inhibitor.

In some embodiments, the menin inhibitor is effective or therapeutically effective at a lower dose when combined with the cellular efflux transporter inhibitor, particularly the P-gp inhibitor and/or the BCRP inhibitor, than without the cellular efflux transporter inhibitor. In some embodiments, the menin inhibitor is more effective (e g, has improved efficacy as described herein) in combination with a cellular efflux transporter inhibitor, particularly a P-gp inhibitor and/or a BCRP inhibitor, than at the same dose but without the cellular efflux transporter inhibitor.

In some embodiments, the present disclosure provides a method for increasing absolute neutrophil count (ANC) in a subject with an acute leukemia, such as AML, comprising administering to the subject a menin inhibitor (e.g., Compound I) and a cellular efflux transporter inhibitor, particularly (a) a P-gp inhibitor or (b) a BCRP inhibitor. In some embodiments, the subject is already being treated with the menin inhibitor prior to initiation of administration of the cellular efflux transporter inhibitor. In some embodiments, the subject's ANC was increasing prior to initiation of administration of the cellular efflux transporter inhibitor.

The present disclosure provides a method for increasing bioavailability of a menin inhibitor. The method may comprise administering to the subject a menin inhibitor (e.g., Compound I) and a cellular efflux transporter inhibitor, particularly (a) a P-gp inhibitor or (b) a BCRP inhibitor.

In some embodiments, a cellular efflux transporter inhibitor, particularly a P-gp inhibitor or a BCRP inhibitor is administered in an amount such that the bioavailability of the menin inhibitor is increased by between about 10% to about 1000% as compared to administration of the menin inhibitor in the absence of the cellular efflux transporter inhibitor. In some embodiments, a cellular efflux transporter inhibitor, particularly a P-gp inhibitor or a BCRP inhibitor, is administered in an amount such that the bioavailability of the menin inhibitor is increased by at least 20%, 30%, 40%, 50%, 60%, 70%, 80%, 90% 100%, 200%, 300%, or 400% as compared to administration of the menin inhibitor in the absence of the cellular efflux transporter inhibitor. In some embodiments, a cellular efflux transporter inhibitor, particularly a P-gp inhibitor or a BCRP inhibitor, increases bioavailability of the menin inhibitor by at least 2-, 3-, 4-, 5-, 6-, 7-, 8-, 9-, or 10-fold as compared to administration of the menin inhibitor in absence of the cellular efflux transporter inhibitor. In some embodiments, increasing bioavailability comprises one or more of increasing Cₘₐₓ, increasing C_{trough}, increasing area under the curve (AUC), increasing T_{1/2}, and decreasing clearance (Cl). In some embodiments, increasing bioavailability comprises increasing AUC or increasing Cₘₐₓ. In some embodiments, the bioavailability of the menin inhibitor is measured by Area Under the Curve (AUC) or Cₘₐₓ.

In some embodiments of the methods provided herein, when the method comprises administering to the subject a menin inhibitor and a cellular efflux transporter inhibitor, the method further comprises administering to the subject both a P-gp inhibitor and a BCRP inhibitor.

In some embodiments, the dose of the menin inhibitor is adjusted for the administration of the menin inhibitor with a cellular efflux transporter inhibitor, particularly the P-gp inhibitor or BCRP inhibitor, as compared to when the menin inhibitor is administered alone. In some embodiments, the present disclosure provides a method of reducing the dose of a menin inhibitor to a subject who is being treated with a menin inhibitor at an initial dose comprising administering to the subject a cellular efflux transporter inhibitor, particularly (a) a P-gp inhibitor or (b) a BCRP inhibitor, in addition to the menin inhibitor at a dose that is lower than the initial dose.

In some embodiments, a cellular efflux transporter inhibitor, particularly the P-gp inhibitor or the BCRP inhibitor, increases permeability of the menin inhibitor through cell membranes (e.g., of cancer cells) by at least 20%, 30%, 40%, 50%, 60%, 70%, 80%, 90%, 100%, 200%, 300%, or 400% compared to permeability of the menin inhibitor in the absence of the cellular efflux transporter inhibitor.

The present disclosure provides a method for increasing the concentration (e.g., Cₘₐₓ or C_{trough}) of a menin inhibitor in a tissue of a subject receiving treatment with the menin inhibitor comprising administering to the subject a cellular efflux transporter inhibitor, particularly (a) a P-gp inhibitor or (b) a BCRP inhibitor. In some embodiments, a cellular efflux transporter inhibitor, particularly the P-gp inhibitor or the BCRP inhibitor, increases the concentration of the menin inhibitor in a tissue (e.g., in cancer tissue or in brain tissue or in plasma or in bone marrow) by at least 20%, 30%, 40%, 50%, 60%, 70%, 80%, 90%, 100%, 200%, 300%, or 400% compared to the concentration of the menin inhibitor in the tissue in the absence of the cellular efflux transporter inhibitor.

In some embodiments, a cellular efflux transporter inhibitor, particularly a P-gp inhibitor or a BCRP inhibitor, is administered in an amount such that the concentration of the menin inhibitor in a tissue (e.g., in cancer tissue or in brain tissue or in plasma or in bone marrow) is increased by between about 10% to about 1000% as compared to administration of the menin inhibitor in the absence of the cellular efflux transporter inhibitor. In some embodiments, a cellular efflux transporter inhibitor, particularly a P-gp inhibitor or a BCRP inhibitor, is administered in an amount such that the concentration of the menin inhibitor in tissues is increased by at least 20%, 30%, 40%, 50%, 60%, 70%, 80%, 90%, 100%, 200%, 300%, or 400% as compared to administration of the menin inhibitor in the absence of the cellular efflux transporter inhibitor. In some embodiments, a cellular efflux transporter inhibitor, particularly a P-gp inhibitor or a BCRP inhibitor, increases the concentration of the menin inhibitor in a tissue by at least 2-, 3-, 4-, 5-, 6-, 7-, 8-, 9-, or 10-fold as compared to administration of the menin inhibitor in absence of the cellular efflux transporter inhibitor. Tissue analysis may be performed on a tissue biopsy sample, bone marrow sample, blood sample, or plasma sample and using methods such as extraction, chromatography, and mass spectrometry.

In some embodiments, a cellular efflux transporter inhibitor, particularly a P-gp inhibitor or a BCRP inhibitor, increases the concentration of the menin inhibitor in cells (e.g., in cancer cells) by at least 20%, 30%, 40%, 50%, 60%, 70%, 80%, 90%, 100%, 200%, 300%, or 400% compared to the concentration of the menin inhibitor in cells in the absence of the cellular efflux transporter inhibitor. Cellular analysis may be performed using a tissue biopsy sample, bone marrow sample, or blood sample and using methods such as dissection, cell separation, and mass spectrometry. In some embodiments, the cellular efflux transporter inhibitor, particularly the P-gp inhibitor, reduces transport of the menin inhibitor from one or more cells of the subject.

In some embodiments, the cellular efflux transporter inhibitor, particularly the P-gp inhibitor or BCRP inhibitor, is administered in an amount such that the permeability of the menin inhibitor through cell membranes (e.g., of cancer cells) is increased by between about 10% to about 1000% as compared to administration of the menin inhibitor in the absence of the cellular efflux transporter inhibitor. In some embodiments, the cellular efflux transporter inhibitor, particularly the P-gp inhibitor or BCRP inhibitor, is administered in an amount such that the permeability of the menin inhibitor through cell membranes is increased by at least 20%, 30%, 40%, 50%, 60%, 70%, 80%, 90%, 100%, 200%, 300%, or 400% as compared to administration of the menin inhibitor in the absence of the cellular efflux transporter inhibitor. In some embodiments, the cellular efflux transporter inhibitor, particularly the P-gp inhibitor or BCRP inhibitor, increases the permeability of the menin inhibitor through cell membranes by at least 2-, 3-, 4-, 5-, 6-, 7-, 8-, 9-, or 10-fold as compared to administration of the menin inhibitor in the absence of the cellular efflux transporter inhibitor.

In some embodiments, an effective amount, such as a therapeutically effective amount, of the menin inhibitor is administered. In some embodiments, the effective or therapeutically effective amount of the menin inhibitor is lower than the amount of menin inhibitor that would be administered to the subject without the cellular efflux transporter inhibitor, particularly the P-gp inhibitor or BCRP inhibitor. In some embodiments, the effective or therapeutically effective amount of the menin inhibitor when administered with the cellular efflux transporter inhibitor, particularly the P-gp inhibitor or BCRP inhibitor, is at least 20%, 30%, 40%, 50%, 60%, 70%, 80%, or 90% lower than the effective or therapeutically effective amount of the menin inhibitor without administration of the cellular efflux transporter inhibitor.

In yet another aspect, the present disclosure provides a method of decreasing the cellular efflux transporter activity, particularly the P-gp activity (or BCRP activity), of a subject who is being treated with a menin inhibitor, comprising administering a cellular efflux transporter inhibitor, particularly a P-gp inhibitor (or BCRP inhibitor), to the subject. In some embodiments, the subject expresses P-gp. In some embodiments, the subject overexpresses P-gp relative to a reference level of P-gp expression. In some embodiments, the subject had been administered a menin inhibitor for the treatment of a cancer, such as a hematological malignancy, and developed resistance to the menin inhibitor. In certain embodiments, the subject who has developed resistance has a cancer that has normal level of P-gp expression.

In yet another aspect, the present disclosure provides a method for reducing the risk of developing a resistance to menin inhibitor comprising administering a cellular efflux transporter inhibitor, particularly a P-gp inhibitor or BCRP inhibitor, to the subject.

In yet another aspect, the present disclosure provides a method for modulating the activity of a cellular efflux transporter, particularly P-gp (or BCRP), in a biologic tissue in a subject, such as bone marrow or plasma, the method comprises contacting a tissue in the subject having a P-gp with a pharmaceutically effective amount of a cellular efflux transporter inhibitor, particularly a P-gp inhibitor (or a BCRP inhibitor).

The methods and the compositions provided herein can be used to treat various diseases or disorders treatable by a menin inhibitor (e.g., Compound I), such as a proliferative disease (e.g., a cancer such as a hematological malignancy).

In some embodiments, the hematological malignancy is selected from the group consisting of: Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute lymphocytic leukemia (ALL), mixed phenotype acute leukemia (MPAL), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma (CML), and chronic monocytic leukemia.

In some embodiments, the hematological malignancy may be AML, ALL, or MPAL. In some embodiments, the subject exhibits a rearrangement in the KMT2A(MLL) gene. In some embodiments, the subject does not exhibit a mutation in the mixed-lineage leukemia (MLL) gene. In some embodiment, the hematological malignancy is relapsed or refractory. In some embodiments, the hematologic malignancy may exhibit a mutation in the NPM1 gene, such as a Type A, B, or D mutation. In some embodiments, the AML, ALL, or MPAL is relapsed or refractory AML, ALL, or MPAL. In one embodiment, the hematological malignancy is relapsed or refractory AML.

In some embodiments, the hematological malignancy is MM. In some embodiments, the hematological malignancy is CLL. In some embodiments, the hematological malignancy is DLBCL. In some embodiments, the hematological malignancy is MDS. In some embodiments, the hematological malignancy is CML.

In some embodiments, the hematological malignancy is relapsed or refractory.

In another aspect, the present disclosure provides use of a menin inhibitor (e.g., Compound I) for treatment of hematological malignancy in a subject in need thereof comprising administering the menin inhibitor to the subject, wherein the subject is concurrently administered a cellular efflux transporter inhibitor, particularly a P-gp inhibitor or a BCRP inhibitor.

### Pharmaceutical Compositions

The pharmaceutical compositions of the present disclosure may be utilized in methods described herein, such as in methods to treat a subject in need of such treatment. Pharmaceutical compositions include at least one pharmaceutically acceptable carrier or excipient and a menin inhibitor (e.g., Compound I), sometimes referred to herein as an active agent or ingredient. In yet another aspect, the present disclosure provides a pharmaceutical composition comprising a menin inhibitor (e.g., Compound I) for use in treating a hematological malignancy in a subject in need thereof administering the menin inhibitor a patient who is concurrently administered a cellular efflux transporter inhibitor, particularly a P-gp inhibitor or a BCRP inhibitor.

In certain embodiments, the subject is a mammal such as a human, or a non-human mammal. When administered to an animal, such as a human, the composition or the compound is administered as a pharmaceutical composition comprising, for example, a menin inhibitor as described herein, and a pharmaceutically acceptable carrier or excipient. In a preferred embodiment, the menin inhibitor is administered as a pharmaceutical composition comprising Compound I, or a pharmaceutically acceptable form thereof, and a pharmaceutically acceptable carrier or excipient.

The menin inhibitor may be in free-acid or free-base form, or in a pharmaceutically acceptable salt form. Additionally, a menin inhibitor (e.g., Compound I) may be in unsolvated or solvated forms with pharmaceutically acceptable solvents such as water and ethanol.

A pharmaceutical composition, as used herein, generally refers to a mixture of a menin inhibitor (e.g., Compound I) with other chemical components, such as carriers, stabilizers, diluents, dispersing agents, suspending agents, thickening agents, wetting or emulsifying agents, solution promoters, salts for regulating osmotic pressure, buffers, and/or excipients. In certain embodiments, the pharmaceutical composition facilitates administration of the compound to an organism. In some embodiments, practicing the methods of treatment or use provided herein, therapeutically effective amounts of a menin inhibitor (e.g., Compound I) are administered in a pharmaceutical composition to a mammal having a hematological malignancy.

In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Suitable routes of administration include, but are not limited to, oral, intravenous, rectal, aerosol, parenteral, ophthalmic, pulmonary, transmucosal, transdermal, vaginal, otic, nasal, and topical administration. In some embodiments, the pharmaceutical composition is formulated for oral administration. In addition, by way of example only, parenteral delivery includes intramuscular, subcutaneous, intravenous, intramedullary injections, as well as intrathecal, direct intraventricular, intraperitoneal, intralymphatic, and intranasal injections. In other embodiments, the pharmaceutical composition is formulated for injection. The exact route of administration, dose, or frequency of administration would be readily determined by those skilled in the art and can be dependent on the age, weight, general physical condition, or other clinical symptoms specific to the patient to be treated. Further exemplary pharmaceutical compositions include: aqueous solutions, for example, formulated in a physiologically compatible buffer, such as Hank's solution, Ringer's solution, or physiological saline buffer; formulations for transmucosal administration, for example, including suitable penetrants; or formulations for parenteral injection, for example, in an aqueous or nonaqueous solution, such as a physiologically compatible buffer and/or excipient; formulations for oral administration, for example, formulated in oral dosage forms that include, by way of example only, tablets, powders, pills, dragees, capsules, liquids, gels, syrups, elixirs, slurries, suspensions and the like; formulations for buccal or sublingual administration, such as tablets, lozenges, or gels; formulations for parental injection, such as bolus injection or continuous infusion, optionally in unit dosage form or in multi-dose containers; formulations for topical administration, such as solutions, suspensions, lotions, gels, pastes, medicated sticks, balms, creams, or ointments; formulations for transdermal administration, optionally employing transdermal delivery devices or patches, lipophilic emulsions, or aqueous solutions; formulations for inhalation, such as aerosols, mists, or powders; or formulations for rectal administration, such as enema solutions, rectal gels, foams, or aerosols, or suppositories.

Pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-(hydroxymethyl)aminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Pharmaceutical compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Pharmaceutical compositions may include one or more antioxidants to enhance chemical stability where required. Suitable antioxidants include, by way of example only, ascorbic acid and sodium metabisulfite.

Pharmaceutical preparations for oral use include suitable excipients, such as in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate. In some instances, oral formulations such as dragee cores and tablets include a suitable coating. Other options include push-fit capsules made of gelatin, as well as soft, sealed capsules made of gelatin and a plasticizer, such as glycerol or sorbitol, along with optional fillers such as lactose, binders such as starches, and/or lubricants such as talc or magnesium stearate and, optionally, stabilizers, diluents or suspending agents or stabilizers.

In some embodiments, the menin inhibitor and the cellular efflux transporter inhibitor are administered by different routes. In some embodiments, the menin inhibitor and the cellular efflux transporter inhibitor are administered by the same route. In some embodiments, the menin inhibitor and the cellular efflux transporter inhibitor are each orally administered. In some embodiments, the menin inhibitor and the cellular efflux transporter inhibitor are concurrently administered, e.g., on concurrent schedules or on the same day or at the same time or approximately the same time. In some embodiments, the menin inhibitor and the cellular efflux transporter inhibitor are administered simultaneously, essentially simultaneously or within the same treatment protocol. In some embodiments, the menin inhibitor and the cellular efflux transporter inhibitor are sequentially administered. In some embodiments, the menin inhibitor and the cellular efflux transporter inhibitor are administered at the same time. In some embodiments, the menin inhibitor and the cellular efflux transporter inhibitor are administered in separate dosage forms. In some embodiments, the menin inhibitor and the cellular efflux transporter inhibitor are administered in a combined dosage form. In some embodiments, the cellular efflux transporter inhibitor is a P-gp inhibitor. In some embodiments, the cellular efflux transporter inhibitor is a BCRP inhibitor.

In some embodiments, the menin inhibitor (e.g., Compound I) is administered in multiple doses. In some embodiments, dosing is about once, twice, three times, four times, five times, six times, or more than six times per day, optionally once or twice per day. In other embodiments, dosing is about once a month, once every two weeks, once a week, or once every other day. In another embodiment, the menin inhibitor (e.g., Compound I) and a second agent (e.g., a P-gp inhibitor) are administered together about once per day to about 6 times per day, or on different schedules (e.g., once a day for the menin inhibitor and once or twice a day for the second agent). In another embodiment, the administration of the menin inhibitor (e.g., Compound I) and a second agent (e.g., a P-gp inhibitor) continues for less than about 7 days. In yet another embodiment, the administration continues for more than about 6 days, more than about 10 days, more than about 14 days, more than about 28 days, more than about two months, more than about six months, or one year or more. In some embodiments, the administration is for a cycle of 28 days or multiple cycles of 28 days. In some cases, continuous dosing is achieved and maintained for as long as necessary.

Administration of a menin inhibitor (e.g., Compound I) may continue as long as necessary. In some embodiments, a compound of the disclosure is administered for more than 1, more than 2, more than 3, more than 4, more than 5, more than 6, more than 7, more than 14, or more than 28 days. In some embodiments, a compound of the disclosure is administered 28 days or less, 14 days or less, 7 days or less, 6 days or less, 5 days or less, 4 days or less, 3 days or less, 2 days or less, or 1 day or a part thereof. In some embodiments, a menin inhibitor (e.g., Compound I) is administered chronically on an ongoing basis, e.g., for the treatment of chronic effects.

### Dosages and Treatment Regimens

In certain embodiments, dosages, treatment regimens, and therapeutically effective amounts vary depending on the severity of the disease, the age and relative health of the subject, the potency of the compound(s) used and other factors.

In some embodiments, the amount of the menin inhibitor administered in the methods provided herein is from 50 mg/day up to, and including, 1000 mg/day.

In some embodiments, the daily dosage of the menin inhibitor is between about 50 mg to about 800 mg. In some embodiments, the daily dosage of the menin inhibitor is about 50 mg. In some embodiments, the daily dosage of the menin inhibitor is about 100 mg. In some embodiments, the daily dosage of the menin inhibitor is about 200 mg. In some embodiments, the daily dosage of the menin inhibitor is about 400 mg. In some embodiments, the daily dosage of the menin inhibitor is about 600 mg. In some embodiments, the daily dosage of the menin inhibitor is about 800 mg.

In some embodiments, a daily dose is given once a day, or is divided and given twice a day, three times per day, four times per day to equal the daily dose. In some embodiments, the menin inhibitor is administered at a unit dose of 25 mg, 50 mg, or 200 mg. In some embodiments, the unit dose is given once a day, given twice a day, given three times per day, given four times per day. In some embodiments, one unit dose is given per day, two unit doses are given per day, three unit doses are given per day, or four unit doses are given per day. In some embodiments, two unit doses are given twice per day. In some embodiments, three unit doses are given once per day. In some embodiments, four unit doses are given once per day.

In some embodiments, the amount of the menin inhibitor that is administered is about 40 mg/day. In some embodiments, the amount of the menin inhibitor that is administered is about 50 mg/day. In some embodiments, the amount of the menin inhibitor that is administered is about 60 mg/day. In some embodiments, the amount of the menin inhibitor that is administered is about 70 mg/day. In some embodiments, the amount of the menin inhibitor that is administered is about 80 mg/day. In some embodiments, the amount of the menin inhibitor that is administered is about 90 mg/day. In some embodiments, the amount of the menin inhibitor that is administered is about 100 mg/day. In some embodiments, the amount of the menin inhibitor that is administered is about 110 mg/day. In some embodiments, the amount of the menin inhibitor that is administered is about 120 mg/day. In some embodiments, the amount of the menin inhibitor that is administered is about 130 mg/day. In some embodiments, the amount of the menin inhibitor that is administered is about 140 mg/day. In some embodiments, the amount of the menin inhibitor that is administered is about 150 mg/day. In some embodiments, the amount of the menin inhibitor that is administered is about 160 mg/day. In some embodiments, the amount of the menin inhibitor that is administered is about 170 mg/day. In some embodiments, the amount of the menin inhibitor that is administered is about 180 mg/day. In some embodiments, the amount of the menin inhibitor that is administered is about 190 mg/day. In some embodiments, the amount of the menin inhibitor that is administered is about 200 mg/day. In some embodiments, the amount of the menin inhibitor that is administered is about 250 mg/day. In some embodiments, the amount of the menin inhibitor that is administered is about 300 mg/day. In some embodiments, the amount of the menin inhibitor that is administered is about 350 mg/day. In some embodiments, the amount of the menin inhibitor that is administered is about 360 mg/day. In some embodiments, the amount of the menin inhibitor that is administered is about 400 mg/day. In some embodiments, the amount of the menin inhibitor that is administered is about 450 mg/day. In some embodiments, the amount of the menin inhibitor that is administered is about 500 mg/day. In some embodiments, the amount of the menin inhibitor that is administered is about 550 mg/day. In some embodiments, the amount of the menin inhibitor that is administered is about 600 mg/day. In some embodiments, the amount of the menin inhibitor that is administered is about 800 mg/day. In some embodiments, the menin inhibitor is SNDX-5613 and the amount administered is 75 mg, 113 mg, 163 mg, 164 mg, or 226 mg once or twice per day. In some embodiments, the menin inhibitor is Compound C and the amount administered is 25, 50, 75, 100, 15, 175, 200, 325, 500, or 650 mg once per day.

In some embodiments, the daily dose of the cellular efflux transport inhibitor that is administered in combination with a menin inhibitor is from 50 mg/day up to, and including, 1000 mg/day. In some embodiments, each dose is given once a day, given twice a day, given three times per day, or given four times per day. In some embodiments, the cellular efflux transport inhibitor dosage is dependent on the specific cellular efflux transport inhibitor. In some embodiments, the daily dosage of each cellular efflux transport inhibitor is administered according to approved labeling for other indications. In some embodiments, the amount of the cellular efflux transport inhibitor that is administered is about 50 mg/day, or about 100 mg/day, or about 150 mg/day, or about 200 mg/day, or about 250 mg/day, or about 300 mg/day, or about 350 mg/day, or about 400 mg/day, or about 500 mg/day. In some embodiments, each dose is given once a day, given twice a day, given three times per day, or given four times per day. In some embodiments, the cellular efflux transporter inhibitor is a P-gp inhibitor. In some embodiments, the P-gp inhibitor is ketoconazole, and the amount is 200 mg, 400 mg, 200 to 400 mg once daily, or 200 mg once daily. In some embodiments, the P-gp inhibitor is itraconazole and the amount is 130 mg, 200 mg, 260 mg, or 400 mg, or 130 mg QD, or 200 mg QD, or 260 mg QD, or 400 mg QD, or 200 mg TID for three days followed by 200 mg QD, or 130 mg QD, or 130 mg TID for three days followed by 130 mg QD. In some embodiments, the P-gp inhibitor is posaconazole, and the amount is 100 mg, 200 mg, 300 mg, 100 mg QD, 100 mg BID, 200 mg QD, 200 mg BID, 300 mg QD, 300 mg BID, or 100 mg BID for one day followed by 100 mg QD, or 200 mg BID for one day followed by 200 mg QD, or 300 mg BID for one day followed by 300 mg QD. In some embodiments, the cellular efflux transporter inhibitor is a BCRP inhibitor.

### Kits

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

In yet another aspect, the present disclosure provides a kit comprising (1) a therapeutically effective amount of a pharmaceutical composition comprising a menin inhibitor and a pharmaceutically acceptable carrier or excipient, in a first dosage form; and (2) a composition comprising a cellular efflux transporter inhibitor, particularly a P-gp inhibitor or a BCRP inhibitor, and a pharmaceutically acceptable carrier or excipient, in a second dosage form.

The articles of manufacture provided herein contain packaging materials. Packaging materials for use in packaging pharmaceutical products include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes a menin inhibitor (e.g., Compound I), or a pharmaceutically acceptable form thereof, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms containing a compound provided herein. The pack, for example, contains metal or plastic foil, such as a blister pack. The pack or dispenser device may be accompanied by instructions for administration. The pack or dispenser may be accompanied by a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### EXAMPLES

### Example 1: P-Gp Substrate Assessment of Compound I Using MDR1-MDCK Monolayers

MDR1-MDCK cell monolayers were grown to confluence on collagen-coated, microporous membranes in 12-well assay plates. Details of the plates and their certification are shown below. The permeability assay buffer was Hanks' balanced salt solution (HBSS) containing 10 mM HEPES and 15 mM glucose at a pH of 7.4. The buffer in the receiver chamber also contained 1% bovine serum albumin. The dosing solution concentration was 5 μM of test article in the assay buffer+/−1 μM valspodar. Cells were first pre-incubated for 30 min with HBSS containing+/−1 μM valspodar. Cell monolayers were dosed on the apical side (A-to-B) or basolateral side (B-to-A) and incubated at 37° C. with 5% CO₂ in a humidified incubator. Samples were taken from the donor and receiver chambers at 120 min. Each determination was performed in duplicate. The flux of lucifer yellow was also measured post-experimentally for each monolayer to ensure no damage was inflicted to the cell monolayers during the flux period. All samples were assayed by LC-MS/MS using electrospray ionization. Liquid chromatography conditions are summarized as follows: Column: Thermo BDS Hypersil C18 30×2.1 mm, 3 m, with guard column; M.P. Buffer: 25 mM ammonium formate buffer, pH 3.5; Aqueous Reservoir (A): 90% water, 10% buffer; Organic Reservoir (B): 90% acetonitrile, 10% buffer; Flow Rate: 350 μL/minute; Total run time: 3.5 min; Autosampler: 10 μL injection volume; Autosampler wash: water/methanol/2-propanol: 1/1/1; with 0.2% formic acid.

### Gradient Program

| Time (min) | % A | % B |
|---|---|---|
| 0.0 | 100 | 0 |
| 0.5 | 50 | 50 |
| 1.0 | 0 | 100 |
| 1.5 | 0 | 100 |
| 1.6 | 100 | 0 |
| 2.5 | 100 | 0 |

### Cell Bath Quality Control Results

| Plates | 12-well | |
|---|---|---|
| Passage number | 15 | |
| Age at QC (days) | 7 | |
| Age at experiment (days) | 11 | Acceptance criteria |
| TEER value (Ω*cm²) | 1667 | ≥1400 |
| Atenolol Pₐₚₚ, 10⁻⁶ cm/s | 0.05 | ≤0.5 |
| Propanolol Pₐₚₚ, 10⁻⁶ cm/s | 15.0 | 10-30 |
| Digoxin A-to-B Pₐₚₚ, 10⁻⁶ cm/s | 0.07 | ≤0.1 |
| Dixogin B-to-A Pₐₚₚ, 10⁻⁶ cm/s | 17.6 | None |
| Digoxin efflux ratio | 243 | ≥100 |

The apparent permeability (Pₐₚₚ) and percent recovery were calculated as follows:

$${P}_{app} = ( dCr / dt ) \times {V}_{r} / ( A \times {C}_{A} ) \qquad ( 1 )$$

$$Percent Recovery = 100 \times ( ( {V}_{r} \times {C}_{r}^{final} ) + ( {V}_{d} \times {C}_{d}^{final} ) ) / ( {V}_{d} \times {C}_{N} ) \qquad ( 2 )$$

where,
dCr/dt is the slope of the cumulative concentration in the receiver compartment versus time in μM s⁻¹;
Vᵣ is the volume of the receiver compartment in cm³;
V_{d} is the volume of the donor compartment in cm³;
A is the area of the insert (1.13 cm² for 12-well);
C_{A} is the average of the nominal dosing concentration and the measured 120 minute donor concentration in μM;
C_{N} is the nominal concentration of the dosing solution in μM;
Cᵣ^{final} is the cumulative receiver concentration in μM at the end of the incubation period;
C_{d}^{final} is the concentration of the donor in μM at the end of the incubation period.

Efflux ratio (ER) is defined as Pₐₚₚ (B-to-A)/Pₐₚₚ (A-to-B).

The experimental results are summarized in Table 1 below.

TABLE 1

| Test | | Recovery | Pₐₚₚ (10⁻⁶ cm/s) | | | | Efflux | P-gp Substrate |
|---|---|---|---|---|---|---|---|---|
| Article | Direction | (%) | R1 | R2 | R3 | Avg | Ratio | Classification |
| Cmpd I | A-to-B | 70 | 0.09 | 0.09 | 0.09 | 0.09 | 105 | Positive |
| | B-to-A | 83 | 6.43 | 9.21 | 12.6 | 9.41 | | |
| Cmpd I + | A-to-B | 53 | 0.41 | 0.88 | 0.76 | 0.68 | 0.3 | |
| valspodar | B-to-A | 118 | 0.09 | 0.20 | 0.24 | 0.18 | | |

### P-Gp Substrate Classification:

ER≥2.0 without valspodar, and reduced by ≥50% with valspodar: Positive
ER≥2.0 without valspodar, and reduced by <50% with valspodar: Negative
ER<2.0 without and with valspodar: Negative

As shown by the efflux ratios in Table 2, in view of the P-gp substrate classification above, the experimental results showed that Compound I is a P-gp substrate using MDR1-MDCK cell monolayers.

### Example 2: Evaluation of Compound I as a P-gp Substrate

The potential of Compound I to be a substrate of the P-gp efflux transporter was evaluated using MDR1-MDCK epithelial cell monolayers.

### Mass Spectrometry Method Development

The signal was optimized for each test compound by electrospray ionization (ESI) in positive ionization mode. An MS2 scan was used to optimize the precursor ion mass, and an SIM scan was used to optimize the fragmenter voltage. Product ion analysis was used to identify the best fragment and collision energy for analysis. Following method optimization, a test injection was performed using the column/gradient conditions shown below. An ionization ranking was assigned indicating the compound's ease of ionization.

### HPLC Gradient (Waters LC-MS/MS)

| | Flow Rate | % A | % B |
|---|---|---|---|
| Time (min) | (mL/min) | Mobile Phase | Mobile Phase |
| 0.05 | 0.6 | 99.9 | 0.1 |
| 1.0 | 0.6 | 5 | 95 |
| 1.40 | 0.6 | 5 | 95 |
| 1.41 | 0.6 | 99.9 | 0.1 |
| 1.8 | 0.6 | 99.9 | 0.1 |

Note:
Solution A: H₂O with 0.1% Formic acid;
Solution B: Acetonitrile with 0.1% Formic acid

### Experimental Procedure

MDCK epithelial cells stably transfected with the human MDR1 gene (the gene encoding for the efflux protein P-gp) were seeded on a Multiscreen™ plate (Millipore, MA, USA) and formed a confluent monolayer over 4 days prior to the experiment. To verify the MDR1-MDCK cell monolayers were properly formed, aliquots of the cell buffers were analyzed by fluorescence to determine the transport of the impermeable dye Lucifer Yellow. Any deviations from control values are reported.

For apical to basolateral (A-—B) permeability, Compound I in the absence or presence of 2 μM elacridar (a P-gp inhibitor/BCRP inhibitor) was added to the apical (A) side and the amount of permeation was determined on the basolateral (B) side. For basolateral to apical (B→A) permeability, only Compound I in the absence of 2 μM elacridar was added to the B side and the amount of permeation was determined on the A side. The A-side buffer contained 100 M Lucifer yellow dye, in Transport Buffer (1.98 g/L glucose in 10 mM HEPES, 1× Hanks' Balanced Salt Solution) pH 7.4, while the B-side buffer had Transport Buffer at pH 7.4. MDR1-MDCK cells were incubated with these buffers for 2 h, and the receiver side buffer was removed for analysis by LC-MS/MS.

The rate of passage of the test compounds through this cell monolayer barrier from apical to basolateral (A→B) in this bi-directional transport assay was used to determine the apparent permeability coefficient (Pₐₚₚ). Receiver, donor, and dosing solution concentrations were quantified using a calibration curve prepared in transport buffer solution (pH 7.4). Pₐₚₚ is calculated as

$${P}_{app} = \frac{dQ / dt}{{C}_{0} A} ,$$

where dQ/dt is rate of permeation, C₀ is initial concentration of test agent, and A is the area of monolayer (0.11 cm²). The basolateral to apical (B→A) to apical to basolateral (A→B) apparent permeability coefficient ratio

$${R}_{e} = \frac{{P}_{app} ( B \to A )}{{P}_{app} ( A \to B )}$$

is used to determine the extent of efflux or P-gp substrate specificity of the test compounds. When R_{E}>2 for a tested compound, the tested compound is a potential substrate for P-gp.

Compound I was tested at a concentration of 5 μM. Rantinidine, talinolol, warfarin, and loperamide (±2 μM elacridar) were tested as reference compounds and each was tested at a concentration of 10 μM. The experimental results are summarized in Table 2 below.

TABLE 2
MDR1-MDCK P-gp Substrate Identification

| | | | Mean A→B | Mean B→A | | |
|---|---|---|---|---|---|---|
| Test | Test | Assay | Pₐₚₚ | Pₐₚₚ | Efflux | |
| Article | Conc | Duration | (10⁻⁶ cm s⁻¹) | (10⁻⁶ cm s⁻¹) | Ratio | Comment |
| Loperamide | 10 μM | 2 hr | 0.792 | 61.2 | 78.4 | P-gp |
| | | | | | | Control Substrate |
| Loperamide + | 10 μM | 2 hr | 22.5 | 25.7 | 1.15 | P-gp Control |
| 2 μM Elacridar | | | | | | Substrate + |
| | | | | | | Control Inhibitor |
| Compound I | 5 μM | 2 hr | 0.308 | 1.84 | 3.67 | Low Recovery |
| | | | | | | B → A |
| Compound I + | 5 μM | 2 hr | 0.101 | NR | NC | |
| 2 μM Elacridar | | | | | | |
| Ranitidine | 10 μM | 2 hr | 0.0514 | 4.35 | 105 | Low Permeability |
| | | | | | | Control |
| Talinolol | 10 μM | 2 hr | 0.0950 | 4.22 | 44.7 | P-gp Efflux |
| | | | | | | Control |
| Warfarin | 10 μM | 2 hr | 21.5 | 32.3 | 1.54 | High Permeability |
| | | | | | | Control |

Low recovery may be due to poor aqueous solubility, poor stability, and/or non-specific binding. Permeability rates for compounds with low post assay recovery may be underestimated.
Elacridar (where indicated) was used at a concentration of 2 μM.
NR: Not Reported (for B→A testing in the presence of inhibitor)
NC: Not Calculable
*Note:
A→B Pₐₚₚ value and Efflux Ratio for test compound in absence of inhibitor is reported based on single replicate, n = 1.

As shown by the efflux ratio in Table 2 above, Compound I is a P-gp substrate using MDR1-MDCK cell monolayers because the efflux ratio is greater than 2 without elacridar.

### Example 3: Evaluation of Compound I and its metabolites as P-gp substrates

Compound I and Two Metabolites, Compound IA and Compound IB, were Evaluated in preclinical studies as substrates for one or more cellular efflux pumps. Active efflux of the test compounds by specific cellular pumps was measured in transwell chamber assays using polarized epithelial cells (e.g. MDCK, Caco-2) overexpressing specific transporters (e.g., P-gp, BCRP).

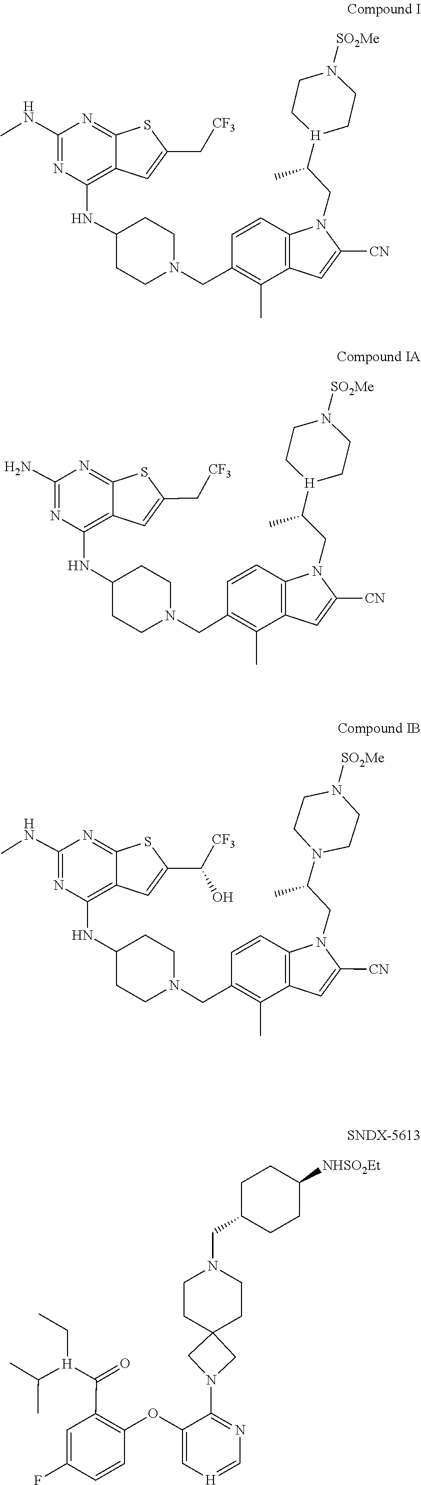

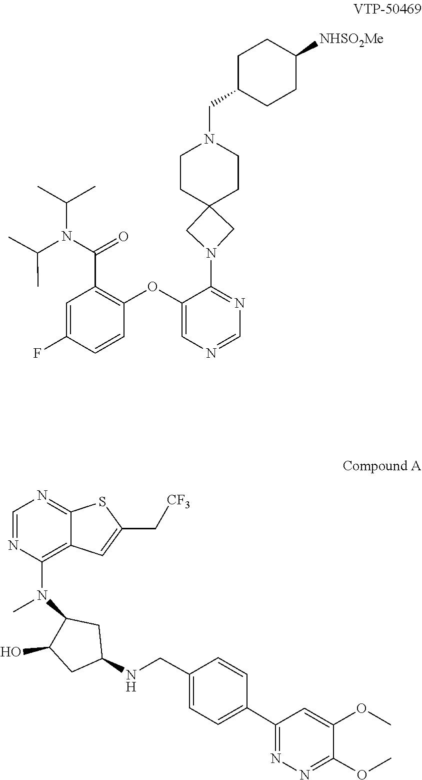

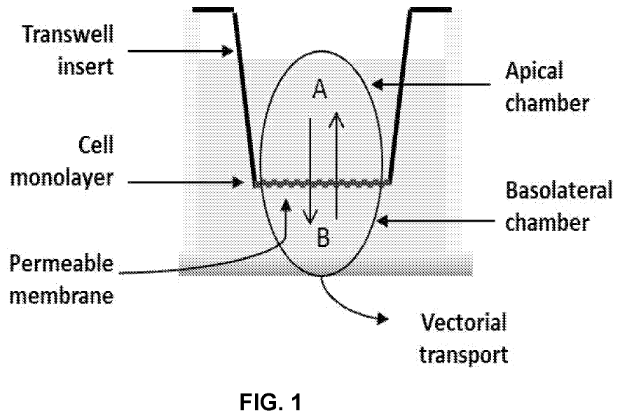

The MDR1-MDCK monolayer was used as an in vitro model for the identification and characterization of P-gp substrates and inhibitors and to predict brain uptake potential. MDR1-MDCK is a cell line originating from Madin Darby canine kidney cells with over expression of MDR1 gene (encoding for P-gp protein) by transfection. The MDCK assay is illustrated in FIG. 1.

The Pₐₚₚ (A→B), Pₐₚₚ (B→A), Efflux ratio, and % Recovery were measured and calculated. The controls were digoxin, with and without a P-gp inhibitor.

### Cell Culture

MDR1-MDCK cells (obtained from the Netherlands Cancer Institute) were seeded onto polyethylene membranes (PET) in 96-well insert systems at 2.5×10⁵ cells/mL until to 4-7 days for confluent cell monolayer formation.

### Experimental Procedures

Test and reference compounds were diluted with transport buffer (HBSS with 10 mM Hepes, pH7.4) from stock solution to a concentration of 2 μM (DMSO<1%) and applied to the apical or basolateral side of the cell monolayer. Permeation of the test compounds and digoxin from A to B direction or B to A direction was determined in duplicate with/without P-gp inhibitor (elacridar (GF120918), 10 μM) and with posaconazole (2 μM and 4 μM, respectively), while nadolol and metoprolol were tested at 2 μM in the absence of elacridar in A to B direction in duplicate. The plate was incubated for 2.5 h in a CO₂ incubator at 37±1° C., with 5% CO₂ at saturated humidity without shaking. In addition, the efflux ratio of each compound was also determined. Test and reference compounds were quantified by LC-MS/MS analysis based on the peak area ratio of analyte/IS.

After the transport assay, Lucifer yellow rejection assay was performed to determine the cell monolayer integrity. Buffers were removed from both apical and basolateral chambers, followed by the addition of 75 μL of 100 μM lucifer yellow in transport buffer and 250 μL transport buffer in apical and basolateral chambers, respectively. The plate was incubated for 30 min at 37° C. with 5% CO₂ and saturated humidity without shaking. After 30 min incubation, 20 μL of lucifer yellow samples are taken from the apical sides, followed by the addition of 60 μL of Transport Buffer. Then, 80 μL of lucifer yellow samples were taken from the basolateral sides. The relative fluorescence unit (RFU) of lucifer yellow was measured at 425/528 nm (excitation/emission) with an Envision plate reader.

### Data Analysis

The apparent permeability coefficient Pₐₚₚ (cm/s) was calculated using the equation:

$${P}_{app} = ( {dC}_{r} / dt ) \times {V}_{r} / ( A \times {C}_{0} )$$

where dCᵣ/dt is the cumulative concentration of compound in the receiver chamber as a function of time (μM/s); Vᵣ is the solution volume in the receiver chamber (0.075 mL on the apical side, 0.25 mL on the basolateral side); A is the surface area for the transport, i.e. 0.0804 cm² for the area of the monolayer; C₀ is the initial concentration in the donor chamber (μM).

The efflux ratio was calculated using the equation:

$$Efflux Ratio = {P}_{app} ( BA ) / {P}_{app} ( AB )$$

Percent recovery was calculated using the equation:

$$\% Recovery = 100 \times [ ( {V}_{r} \times {C}_{r} ) + ( {V}_{d} \times {C}_{d} ) ] / ( {V}_{d} \times {C}_{0} )$$

where V_{d} is the volume in the donor chambers (0.075 mL on the apical side, 0.25 mL on the basolateral side); C_{d} and Cᵣ are the final concentrations of transport compound in donor and receiver chambers, respectively.

Percent of lucifer yellow in basolateral well is calculated using the equation:

$$\% Lucifer Yellow = \frac{{V}_{Basolateral} \times {RFU}_{Basolateral}}{{V}_{Apical} \times {RFU}_{Apical} + {V}_{Basolateral} \times {RFU}_{Basolateral}} \times 100$$

where RFU_{Apical} and RFU_{Basolateral} are the relative fluorescence unit values of lucifer yellow in the apical and basolateral wells, respectively; VApical and VBasolateral are the volume of apical and basolateral wells (0.075 mL and 0.25 mL), respectively. The % Lucifer Yellow should be less than 2.

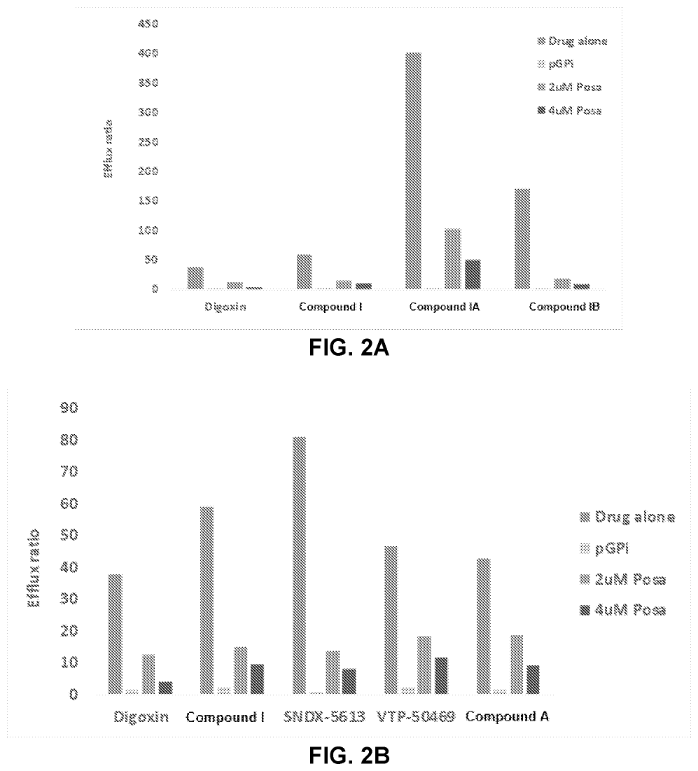

As shown in FIGS. 2A and 2B, Compound I, Compound IA, and Compound IB, as well as the tested reference compounds SNDX-5613, VTP-50469, and Compound A, are substrates of P-gp.

### Example 4: Evaluation of Influence of Efflux Pumps on Antitumor Activity

The influence of efflux pumps on the antitumor activity of the test compounds at steady state (more clinically relevant) were evaluated using P-gp-transfected AML cells (MV4;11 cells) in 6-day viability assays.

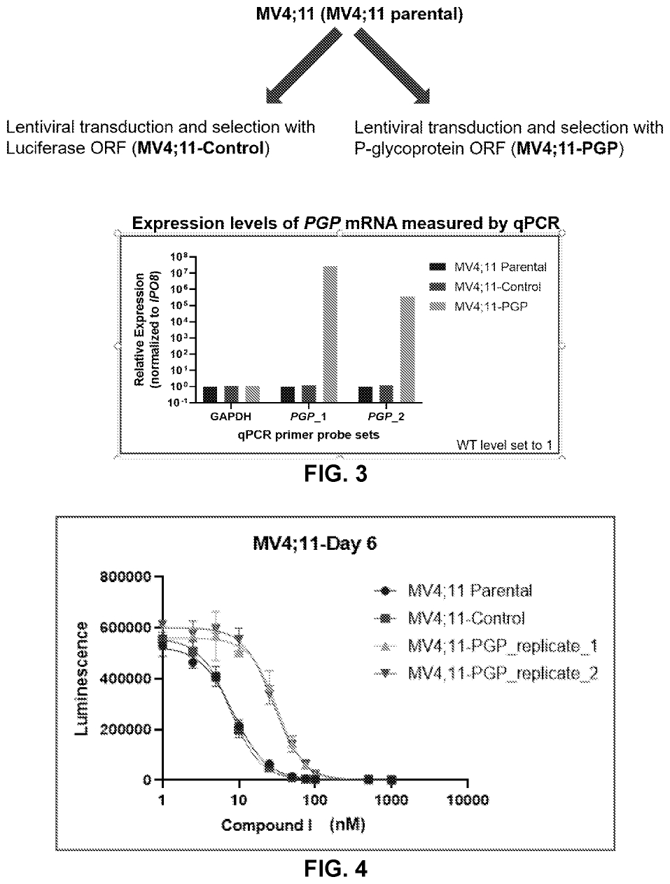

A P-gp-expressing KMT2A(MLL)-rearranged cell line model was developed by lentiviral transduction of the MV4;11 parental cell line and then selection for either Luciferase ORF (MV4;11-Control) or P-gp ORF (MV4;11-PGP). As shown in FIG. 3, the expression levels of PGP mRNA in the MV4;11 parental cell line, MV4;11-Control cell line, and MV4;11-PGP cell line were measured by qPCR to confirm the P-gp expression in MV4;11-PGP cell line (WT level set to 1).

The MV4;11 parental, MV4;11-Control, and MV4;11-PGP cells were seeded on a 96-well plate and the cell lines were treated with Compound I at doses ranging from 0 to 1000 nM for 6 days. Cell viability was measured by CellTiter-Glo® Luminescent Cell Viability Assay (CTG assay, Promega Corp.) and % Viability was quantified by normalizing to the luminescence values of DMSO-only treated cells. The dose-response curves and IC₅₀'s were calculated using GraphPad Prism 9.4.1. The IC₅₀'s of Compound I against the MV4;11 parental, MV4;11-Control, and MV4;11-PGP cell lines are shown in Table 3 and in FIG. 4. The results showed the P-gp expression in cancer cell line (MV4;11 (KMT2A(MLL)-rearranged)) decreases the sensitivity of the cancer cell line to menin inhibitor Compound I, for example, from 8.5 nM (parental) and 7.6 nM (control) to over 25 nM (32.1 nM and 28.5 nM) in two replicates.

TABLE 3

| | MV4; 11 | MV4; 11- | MV4; 11- | MV4; 11- |
|---|---|---|---|---|
| Cell Line | Parental | Control | PGP_replicate_1 | PGP_replicate_2 |
| IC₅₀ (nM) | 8.5 | 7.6 | 32.1 | 28.5 |

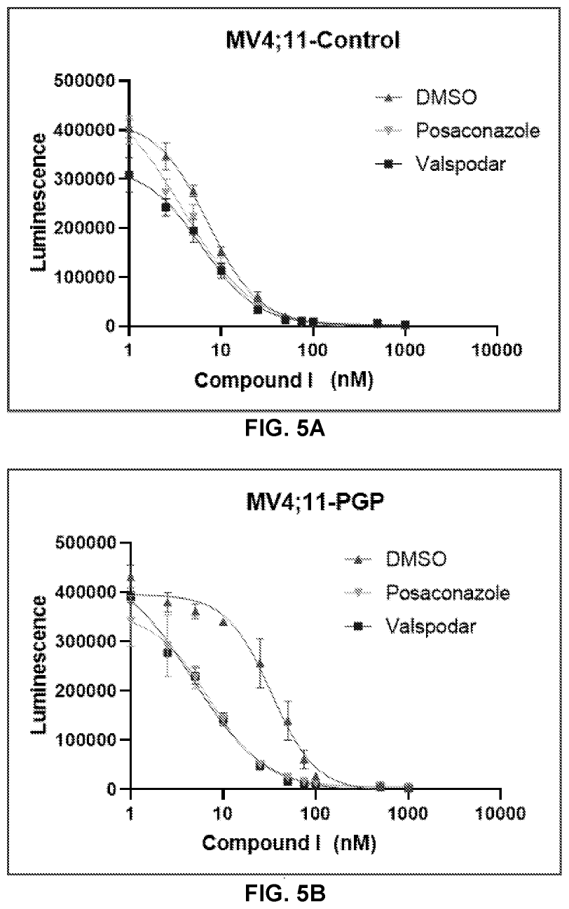

In contrast, when the MV4;11-Control and MV4;11-PGP cell lines were treated with Compound I in combination with a P-gp inhibitor, i.e., posaconazole (1.5 μM) or valspodar (1 μM) for 5 days, the P-gp inhibitor (posaconazole or valspodar) reversed the P-gp-induced sensitivity loss (MV4;11 (KMT2A(MLL)-rearranged)) to Compound I. As shown in Table 4, the IC₅₀'s of Compound I against the MV4;11-PGP cell line decreased to 7.1 nM and 4.7 nM for MV4;11-PGP cells that were co-treated with posaconazole and valspodar, respectively. The measured luminescence in the live cells of the MV4;11-Control and MV4;11-PGP cells that were treated with Compound I only (+DMSO), Compound I+posaconazole (1.5 mM), and valspodar (1 mM) are shown in FIGS. 5A and 5B.

TABLE 4

| | IC₅₀ (Compound | IC₅₀ (Compound | IC₅₀ (Compound |
|---|---|---|---|
| Cell line | I + DMSO) | I + Posaconazole) | I + Valspodar) |
| MV4; 11-Control | 7.2 nM | 3.8 nM | 6.1 nM |
| MV4; 11-PGP | 33 nM | 7.1 nM | 4.7 nM |

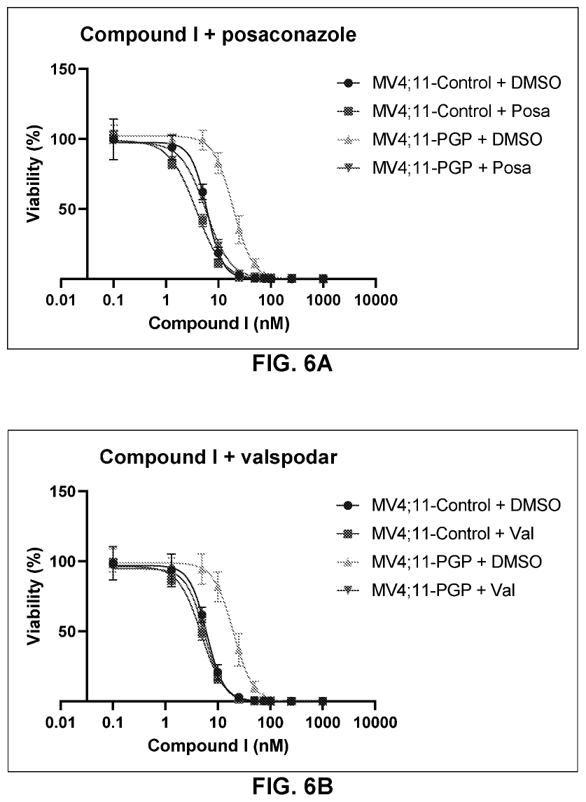

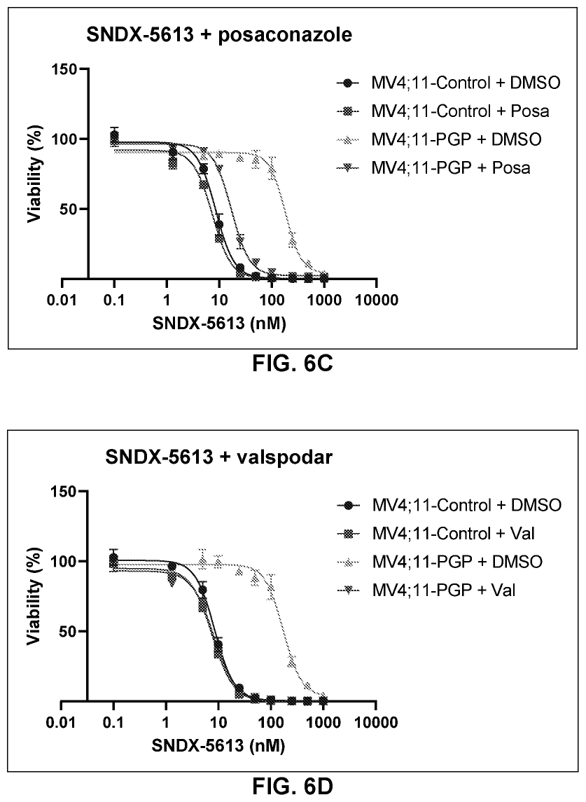

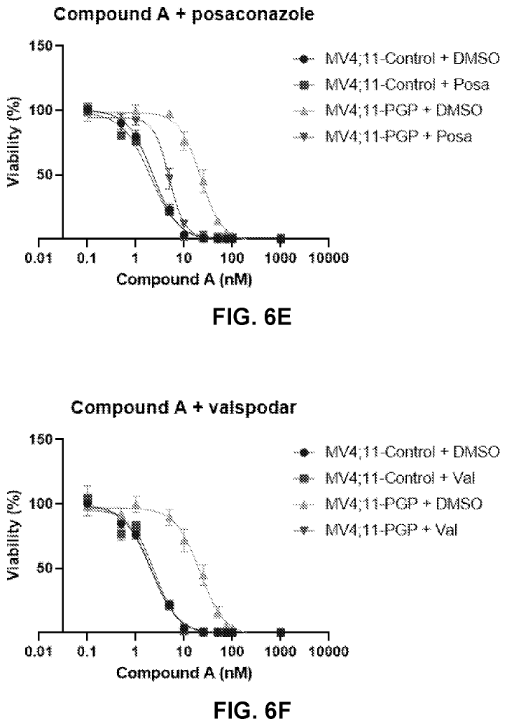

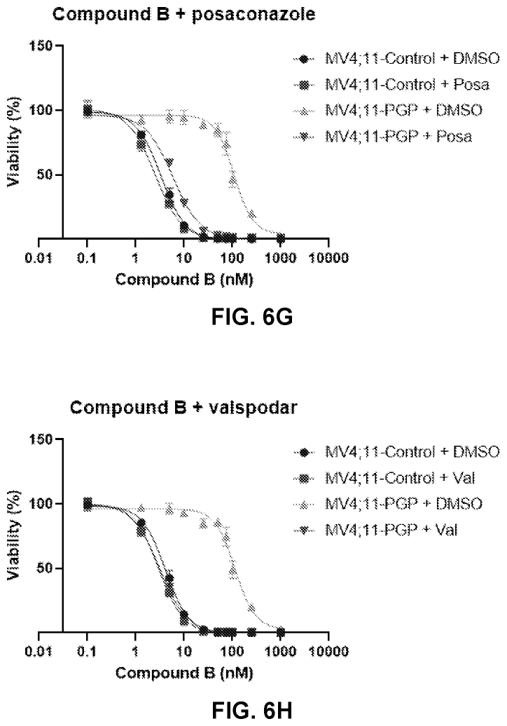

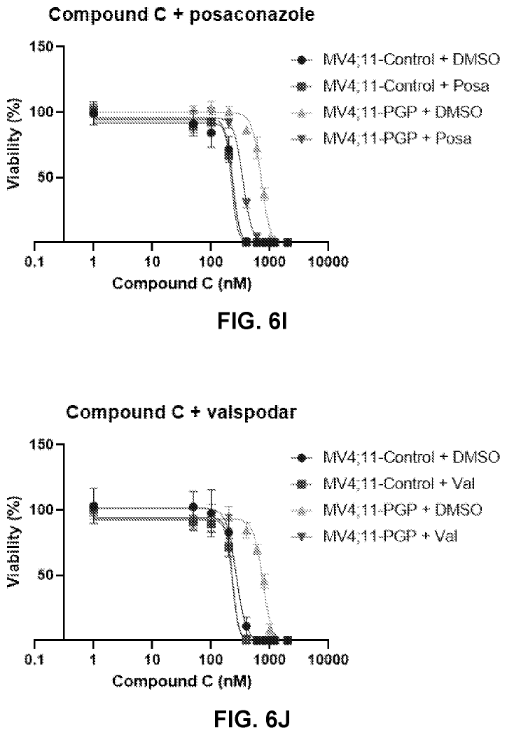

In a second set of experiments, MV4;11-Control and MV4;11-PGP cell lines were treated as described above with five menin inhibitors, including Compound I, SNDX-5613, Compound A, Compound B, and Compound C, at concentrations ranging from 0 to 1000 nM. All tested compounds dropped in potency due to the P-gp expression as shown in FIGS. 6A-6J and in Tables 5 and 6 (indicated by increased IC₅₀ values between “MV4;11-Control (Compound+DMSO)” and “MV4;11-PGP (Compound+DMSO)” entries). Notably, Compound I showed significantly less change in IC₅₀ than the other menin inhibitors tested.

TABLE 5

| | IC₅₀ | IC₅₀ | IC₅₀ | IC₅₀ |
|---|---|---|---|---|
| | MV4; 11- | MV4; | MV4; | MV4; |
| | Control | 11-PGP | 11-Control | 11-PGP |
| | (Compound + | (Compound + | (Compound + | (Compound + |
| | DMSO) | DMSO) | posaconazole) | posaconazole) |
| Compound I | 6.05 nM | 19.1 nM | 3.76 nM | 5.79 nM |
| SNDX-5613 | 8.55 nM | 183 nM | 7.33 nM | 17.1 nM |
| Compound A | 2.28 nM | 22.0 nM | 2.01 nM | 4.91 nM |
| Compound B | 3.32 nM | 108 nM | 2.51 nM | 5.91 nM |
| Compound C | 236 nM | 716 nM | 222 nM | 349 nM |

Co-exposure of the cells to each menin inhibitor and posaconazole (1.5 mM) or valspodar (1 mM) reversed the P-gp-induced sensitivity loss in each case. Specifically, posaconazole reversed the change in IC₅₀ for Compound I and Compound B completely, and partially for the other menin inhibitors (FIGS. 6A, 6C, 6E, 6G, and 6I and Table 5). Co-exposure of the cells to the menin inhibitors and valspodar (used as a control; capable of inhibiting P-gp completely at 1 mM) induced the complete reversal of IC₅₀ erosion for all tested menin inhibitors (FIGS. 6B, 6D, 6F, 6H, and 6J and Table 6).

TABLE 6

| | IC₅₀ | IC₅₀ | IC₅₀ | IC₅₀ |
|---|---|---|---|---|
| | MV4; 11- | MV4; | MV4; | MV4; |
| | Control | 11-PGP | 11-Control | 11-PGP |
| | (Compound + | (Compound + | (Compound + | (Compound + |
| | DMSO) | DMSO) | valspodar) | valspodar) |
| Compound I | 6.14 nM | 19.9 nM | 4.84 nM | 5.64 nM |
| SNDX-5613 | 8.58 nM | 173 nM | 7.61 nM | 8.15 nM |
| Compound A | 2.07 nM | 21.3 nM | 2.10 nM | 2.41 nM |
| Compound B | 4.10 nM | 114 nM | 3.00 nM | 3.15 nM |
| Compound C | 267 nM | 764 nM | 230 nM | 231 nM |

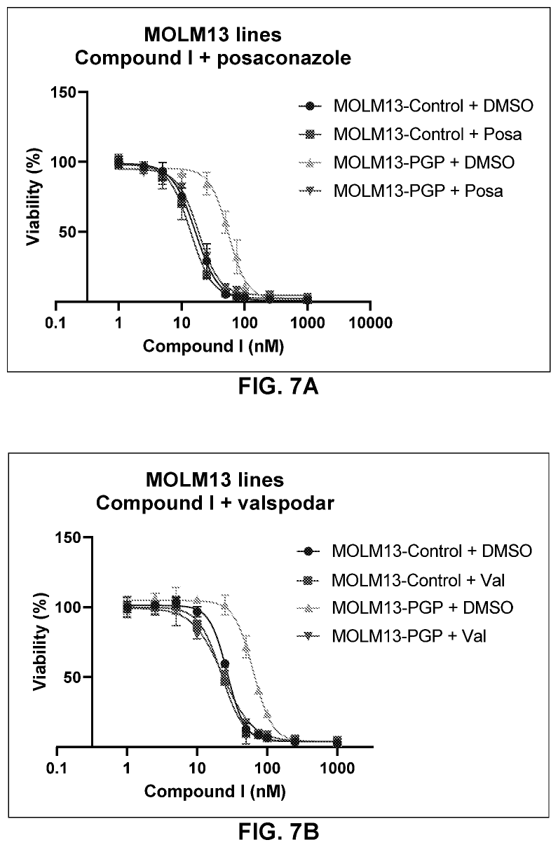

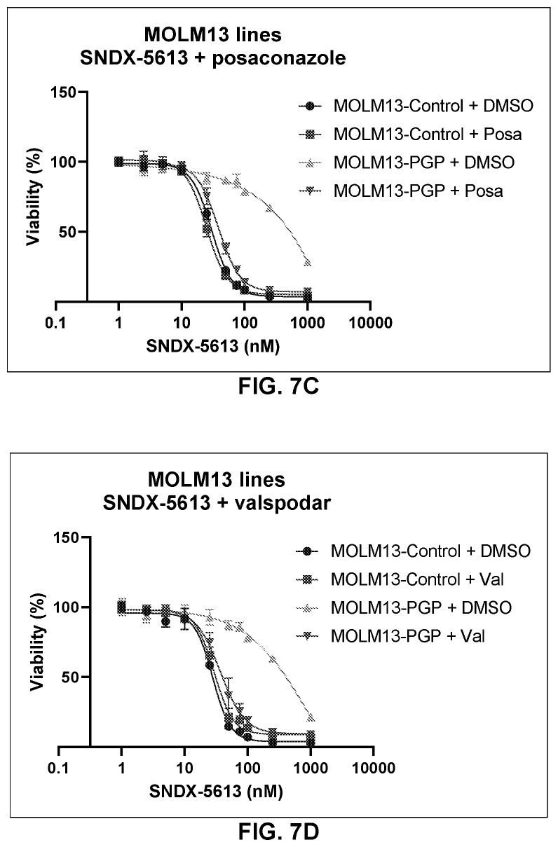

The effects described above were investigated in an AML cell line, MOLM-13, which carries a KA4T2A (MLL)-rearrangement mutation. The P-gp expressing MOLM-13 cell line (MOLM13-PGP) and the Luciferase expressing MOLM-13 cell line (MOLM13-Control) were established by lentivirally transducing P-gp ORF or Luciferase ORF. Cells were plated and treated with test compound for 7 days. After exposure to Compound I or SNDX-5613 for 7 days, the IC₅₀ values for the two test compound significantly increased in the MOLM13-PGP cell line compared to the MOLM13-Control cell line (FIGS. 7A-7D, Tables 7 and 8; compare “MOLM13-Control (Compound+DMSO)” and “MOLM13-PGP (Compound+DMSO)”), suggesting that P-gp expression desensitizes MOLM-13 cells to these compounds. Notably, as observed previously in the MV4;11 system, the impact of P-gp expression on the SNDX-5613 IC₅₀ was much greater than for Compound-I (FIGS. 7A-7D, Tables 7 and 8; compare “MOLM13-Control (Compound+DMSO)” and “MOLM13-PGP (Compound+DMSO)”). Co-exposure of the two test compounds with posaconazole (1.5 mM) or valspodar (1 mM) almost completely reversed the IC₅₀ increases for these two compounds (FIGS. 7A-7D, Tables 7 and 8).

TABLE 7

| | IC₅₀ | IC₅₀ | IC₅₀ | IC₅₀ |
|---|---|---|---|---|
| | MOLM13- | MOLM13- | MOLM13- | MOLM13- |
| | Control | PGP | Control | PGP |
| | (Compound + | (Compound + | (Compound + | (Compound + |
| Cell line | DMSO) | DMSO) | posaconazole) | posaconazole) |
| Compound I | 23.7 nM | 65.7 nM | 16.5 nM | 19.2 nM |
| SNDX-5613 | 30.1 nM | >900 nM | 24.8 nM | 38.6 nM |

TABLE 8

| | IC₅₀ | IC₅₀ | IC₅₀ | IC₅₀ |
|---|---|---|---|---|
| | MOLM13- | MOLM13- | MOLM13- | MOLM13- |
| | Control | PGP | Control | PGP |
| | (compound + | (Compound + | (Compound + | (Compound + |
| Cell line | DMSO) | DMSO) | valspodar) | valspodar) |
| Compound I | 27.0 nM | 63.2 nM | 22.3 nM | 23 nM |
| SNDX-5613 | 28.0 nM | >900 nM | 29.5 nM | 37.1 nM |

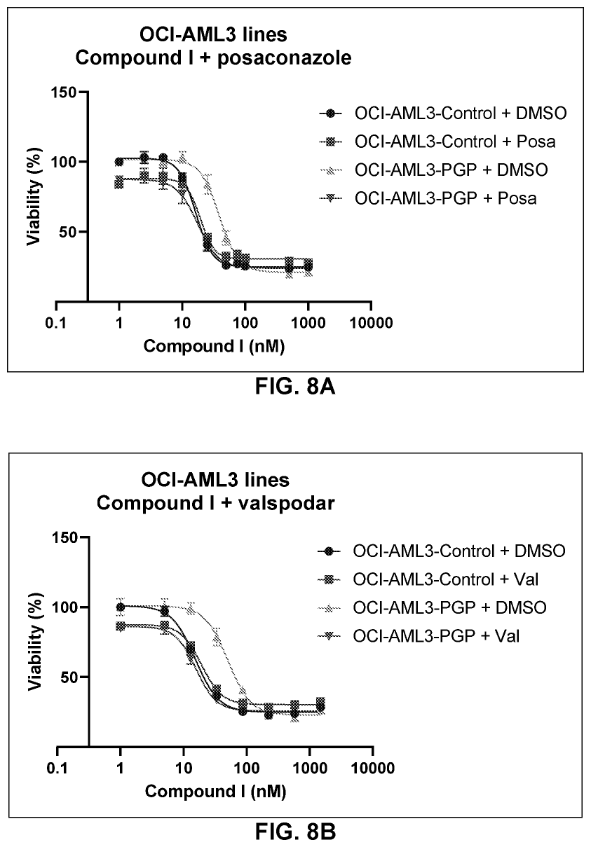

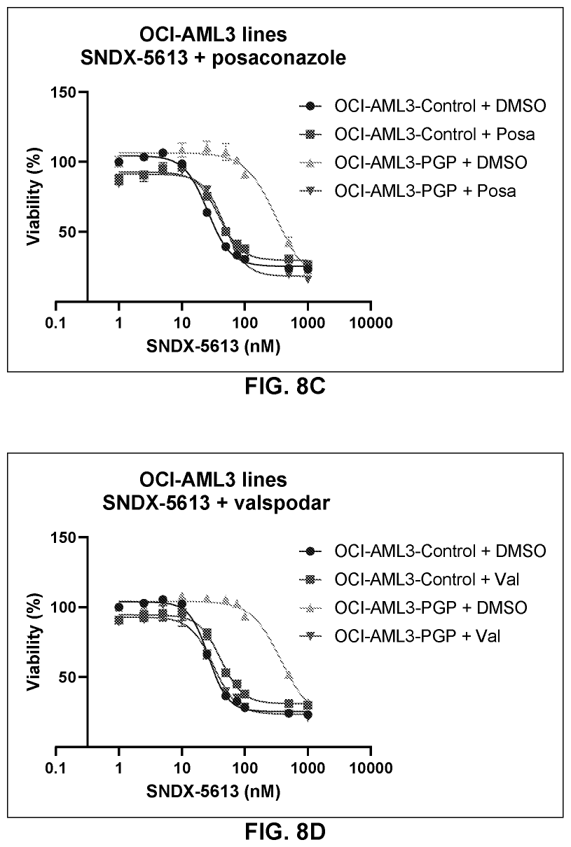

A third set of experiments was conducted in the cell line OCI-AML3, which carries the W288Cfs*12 mutation in NPM protein. The P-gp expressing OCI-AML3 cell line (OCI-AML3-PGP) and the Luciferase expressing OC-AML3 cell line (OCI-AML3-Control) were established by lentivirally transducing P-gp ORF or Luciferase ORF. Cells were treated with Compound I or SNDX-5613 for 7 days to assess the IC₅₀ values. P-gp expression increased the IC₅₀ values of both menin inhibitors in OCI-AML3 cells (FIGS. 8A-8D, Tables 9 and 10; compare “OCI-AML3-Control (Compound+DMSO)” and “OCI-AML3-PGP (Compound+DMSO)”), suggesting that expression of P-gp desensitized OCI-AML3 cells to menin inhibitors, with a greater effect observed for SNDX-5613. Co-exposure with posaconazole (0.75 mM) or valspodar (1 mM) reversed the IC₅₀ increase of both menin inhibitors in P-gp expressing cells (FIGS. 8A-8D, Tables 9 and 10), similarly to the previous KMT2A(MLL)-rearranged models.

TABLE 9

| | IC₅₀ | IC₅₀ | IC₅₀ | IC₅₀ |
|---|---|---|---|---|
| | OCI-AML3- | OCI-AML3- | OCI-AML3- | OCI- |
| | Control | PGP | Control | AML3-PGP |
| | (Compound + | (Compound + | (Compound + | (Compound + |
| Cell line | DMSO) | DMSO) | posaconazole) | posaconazole) |
| Compound I | 16.4 nM | 38.9 nM | 17.7 nM | 19.2 nM |
| SNDX-5613 | 25.7 nM | >300 nM | 38.8 nM | 47.7 nM |

TABLE 10

| | IC₅₀ | IC₅₀ | IC₅₀ | |
|---|---|---|---|---|
| | OCI-AML3- | OCI-AML3- | OCI-AML3- | IC₅₀ |
| | Control | PGP | Control | OCI-AML3-PGP |
| | (Compound + | (Compound + | (Compound + | (Compound + |
| Cell line | DMSO) | DMSO) | valspodar) | valspodar) |
| Compound I | 15.3 nM | 50.2 nM | 19.2 nM | 15.7 nM |
| SNDX-5613 | 26.4 nM | >300 nM | 41.5 nM | 31.7 nM |

### Example 5: Synthesis of Compound IB and Compound IC

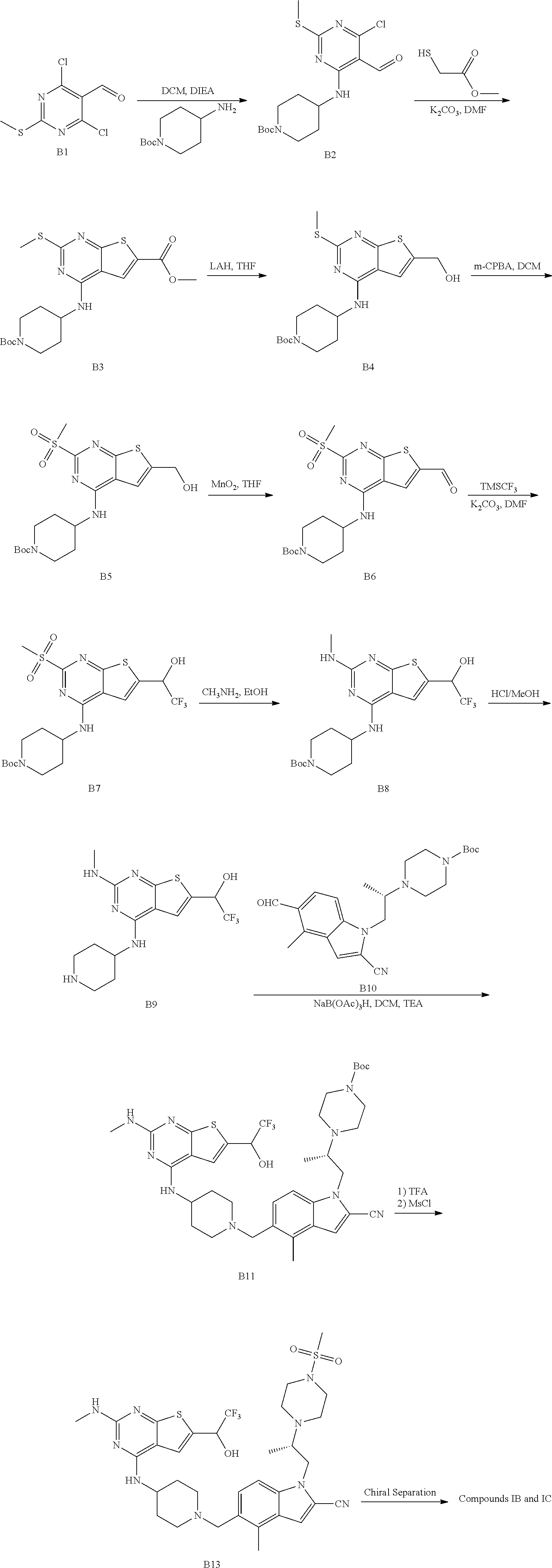

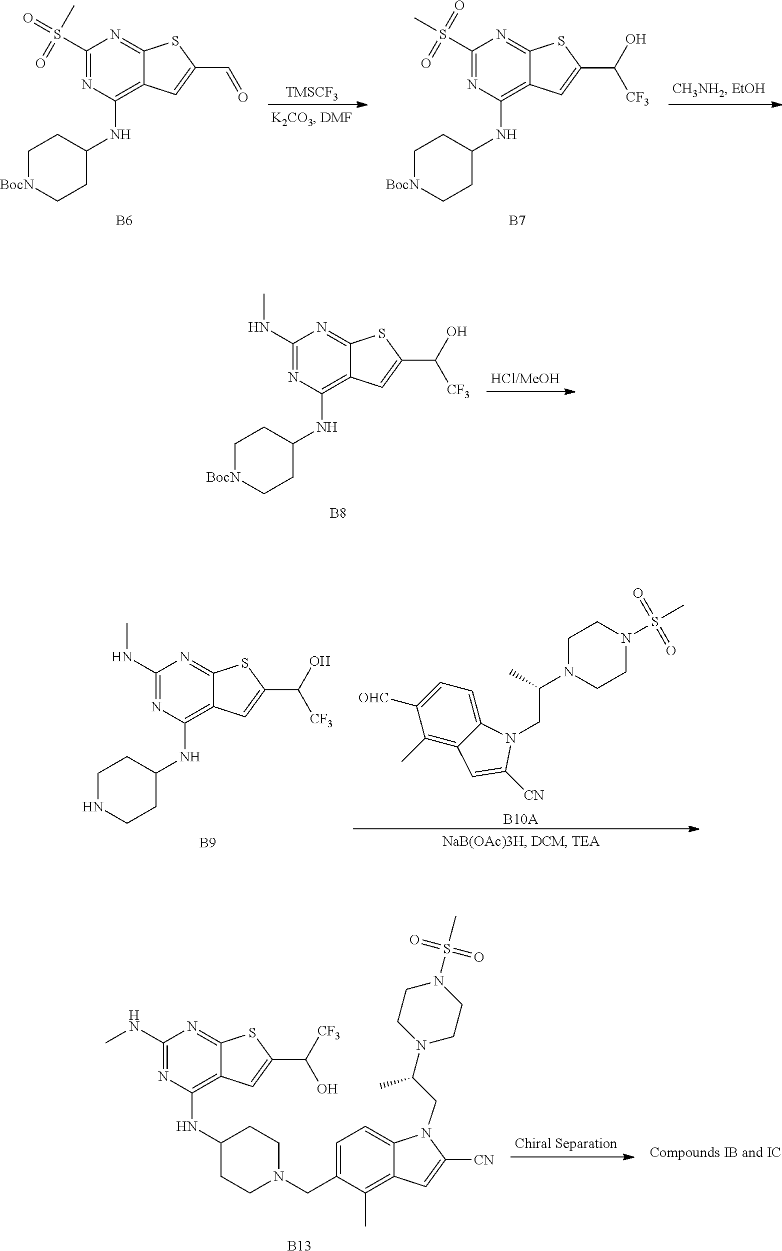

Step A: Preparation of intermediate B2. To a mixture of 4,6-dichloro-2-(methylthio)pyrimidine-5-carbaldehyde (B1, 6.0 g, 27 mmol) and DIEA (4.2 g) in DCM (15 mL) was added tert-butyl 4-aminopiperidine-1-carboxylate (5.6 g, 28 mmol) in DCM (15 mL). The reaction was stirred at room temperature (rt) for 4 h. The reaction mixture was concentrated and purified on silica gel column chromatography to give intermediate B2 (10.0 g, yield: 98%).

Step B: Preparation of intermediate B3. To a mixture of K₂CO₃ (3.93 g, 28.5 mmol) and intermediate B2 (5.5 g, 14.25 mmol) in DMF was added methyl 2-mercaptoacetate (3.0 g, 28.5 mmol) dropwise, and the reaction was stirred at 90° C. for 10 h. The reaction mixture was partitioned between EtOAc and H₂O, and the organic layer was washed with brine and dried over Na₂SO₄. The combined organic layer was concentrated under vacuum to give a residue, which was purified by silica gel column chromatography to give intermediate B3 as a yellow solid (4.6 g, yield: 51%).

Step C: Preparation of intermediate B4. To a solution of intermediate B3 (4.6 g, 10.8 mmol) in THE (100 mL) was added LiAlH₄ (997 mg, 26.25 mmol) at 0° C. for 1 h. The reaction was quenched by 2 N NaOH and Na₂SO₄. The reaction mixture was concentrated under vacuum to give a residue, which was purified by silica gel column chromatography (DCM/MeOH=20:1) to give intermediate B4 (3.2 g, yield: 76.5%).

Step D: Preparation of intermediate B5. To a solution of intermediate B4 (3.2 g, 7.8 mmol) in DCM was added m-CPBA (4.7 g, 23.4 mmol). The reaction was stirred at 0° C. for 10 h. The reaction was quenched with NaHCO₃ (satd. aq.). The reaction mixture was partitioned between DCM and H₂O, and the organic layer was washed by brine and dried over Na₂SO₄. The combined organic layer was concentrated under vacuum to give a residue, which was purified by silica gel column chromatography to give intermediate B5 as a yellow solid (2.0 g, yield: 62%).

Step E: Preparation of intermediate B6. To a solution of intermediate B5 (2.0 g, 4.5 mmol) in THF (20 mL) was added MnO₂ (1.6 g). The reaction was stirred at 90° C. for 24 h. The reaction mixture was concentrated under vacuum to give a residue, which was purified by silica gel column chromatography to give intermediate B6 (1.0 g, yield: 50%).

Step F: Preparation of intermediate B7. To a solution of intermediate B6 (440 mg, 1 mmol) in DMF (10 mL) was added K₂CO₃ (70 mg, 0.5 mmol) at −78° C., then TMSCF₃ (426 mg, 3 mmol) was added. The reaction mixture was stirred at rt for 10 h. The reaction mixture was partitioned between EtOAc and H₂O, and the organic layer was washed with brine and dried over Na₂SO₄. The combined organic layer was concentrated under vacuum to give a residue, which was purified by silica gel column chromatography to give intermediate B7 as a yellow solid (340 mg).

Step G: Preparation of intermediate B8. A mixture of intermediate B7 (400 mg, 1 mmol) in CH₃NH₂ of EtOH (10 mL) was stirred at 100° C. for 10 h. The reaction mixture was concentrated under vacuum to give a residue, which was purified by silica gel column chromatography to give intermediate B8 as a yellow solid (240 mg, yield: 52.1%).

Step H: Preparation of intermediate B9. A solution of intermediate B8 (240 mg, 0.52 mmol) in HCl/MeOH (10 mL) was stirred at rt for 1 h. The reaction mixture was concentrated under vacuum and basified by NH₃·MeOH to give crude intermediate B9, which was used in the next step without further purification.

Step I: Preparation of intermediate B11. A mixture of tert-butyl (S)-4-(1-(2-cyano-5-formyl-4-methyl-1H-indol-1-yl)propan-2-yl)piperazine-1-carboxylate (intermediate B10, see PCT Publ. No. WO2019/060365, p. 148), intermediate B9, and Et₃N in CH₂Cl₂ (10 mL) is stirred at rt for 1 h before NaBH(OAc)₃ (280 mg, 1.32 mmol) is added. The reaction mixture is stirred at rt overnight, then partitioned between CH₂Cl₂ and satd. aq. NaHCO₃. The organic layer is washed with brine and dried over Na₂SO₄. The combined organic layer is concentrated under vacuum to give a residue, which is purified by silica gel column chromatography to give intermediate B11.

Steps J & K: Preparation of Compound B13. Intermediate B11 is treated with TFA in CH₂Cl₂ for Boc deprotection and is then treated with MsCl with Et₃N in CH₂Cl₂ to afford Compound B13 as a white solid. ¹H NMR (400 MHz, DMSO): 7.30-7.51 (m, 5H), 7.16 (d, J=6.8 Hz, 1H), 6.55-6.70 (m, 1H), 5.34 (m, 1H), 4.15-4.36 (m, 2H), 4.02 (br, 1H), 3.50-3.60 (m, 2H), 2.90-3.15 (m, 5H), 2.74-2.84 (m, 10H), 2.52 (s, 3H), 1.45-2.45 (m, 8H), 0.98 (d, J=6.8 Hz, 3H). ESI-MS m/z: 735.25 (M+H).

Alternative Synthesis of Compound B13. A mixture of (S)-5-formyl-4-methyl-1-(2-(4-(methylsulfonyl)piperazin-1-yl)propyl)-1H-indole-2-carbonitrile (intermediate B10A, 243 mg, 0.6 mmol), intermediate B9, and Et₃N (315 mg, 3.1 mmol) in CH₂Cl₂ (20 mL) was stirred at rt for 1.5 h before NaBH(OAc)₃ (664 mg, 3.1 mmol) was added. The reaction mixture was stirred at rt overnight, then was partitioned between CH₂Cl₂ and satd. aq. NaHCO₃. The organic layer was washed with brine and dried over Na₂SO₄. The combined organic layer was concentrated under vacuum to give a residue, which was purified by silica gel column chromatography to give Compound B13 (220 mg, 65%) as a white solid.

Step L: Chiral Separation. Compound B13 was further separated by preparative Supercritical Fluid Chromatography (SFC) to afford diastereomers Compound IB and Compound IC (not shown in scheme). (Instrument: Thar SFC80 preparative SFC; Column: Whelk 0-1, 250*30 mm i.d. 10u; Mobile phase: A for CO₂ and B for IPA (0.1% NH₃/H₂O); Gradient: B %=55%; Flow rate: 85 g/min; Wavelength: 220 nm; Column temperature: 40° C.; System back pressure: 150 bar). Compound IB (100% purity by HPLC): ¹H NMR (400 MHz, CD₃Cl₃) δ (ppm): 7.33 (d, J=8.4 Hz, 1H), 7.16 (s, 1H), 7.07 (d, J=8.4 Hz, 1H), 6.99 (s, 1H), 5.07 (m, 1H), 4.84 (m, 2H), 4.21 (m, 1H), 4.06 (m, 2H), 3.57 (m, 2H), 3.21-3.13 (4H), 2.95 (d, J=4.8 Hz, 3H), 2.86-2.77 (3H), 2.73 (s, 3H), 2.53 (s, 3H), 2.46 (m, 2H), 2.20 (m, 2H), 2.00 (m, 2H), 1.50 (m, 2H), 1.05 (d, J=6.8 Hz, 3H; MS (ESI) m/z 734.2 (MH⁺). Compound IC (100% purity by HPLC): ¹H NMR (400 MHz, CD3Cl₃) δ (ppm): 7.46 (d, J=8.4 Hz, 1H), 7.24 (s, 1H), 7.16 (s, 1H), 7.10 (d, J=8.4 Hz, 1H), 5.07 (m, 1H), 4.85 (m, 2H), 4.19 (m, 1H), 4.05 (m, 2H), 3.57 (m, 2H), 3.21-3.13 (4H), 2.95 (d, J=4.8 Hz, 3H), 2.86-2.77 (3H), 2.73 (s, 3H), 2.54 (s, 3H), 2.47 (m, 2H), 2.20 (m, 2H), 2.00 (m, 2H), 1.50 (m, 2H), 1.05 (d, J=6.8 Hz, 3H); MS (ESI) m/z 734.2 (MH⁺).

### Example 6: Menin Binding

Menin binding was evaluated using an assay analogous to the fluorescence polarization assay described in PCT Publ. No. WO2017/161028, Example 6. IC₅₀ values were determined as follows: Compound IA: 36.2 nM; Compound IB, 63.5 nM; Compound IC, 401 nM).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. Various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for treating a hematological malignancy in a subject in need thereof comprising administering to the subject a menin inhibitor and a cellular efflux transporter inhibitor, optionally selected from a P-glycoprotein (P-gp) inhibitor and a breast cancer resistance protein (BCRP) inhibitor.

2. A method for treating a hematological malignancy in a subject in need thereof who is being treated with a cellular efflux transporter inhibitor comprising administering to the subject a menin inhibitor, wherein the cellular efflux transporter inhibitor is optionally selected from a P-gp inhibitor and a BCRP inhibitor.

3. A method for increasing efficacy of a menin inhibitor for treating a hematological malignancy in a subject in need thereof comprising administering to the subject a menin inhibitor and a cellular efflux transporter inhibitor, optionally selected from a P-gp inhibitor or a BCRP inhibitor, optionally wherein efficacy is measured by one or more of CR (MRD+/−), CRh, CR/CRh, CRi, CRp, CRC, ORR, MLFS, PR, SD, OS, blast count levels, DOR, time to relapse, event-free survival, or progression-free survival, and ANC.

4. A method for increasing bioavailability of a menin inhibitor in a subject comprising administering to the subject the menin inhibitor and a cellular efflux transporter inhibitor, optionally selected from a P-gp inhibitor and a BCRP inhibitor, optionally wherein the subject has a hematological malignancy.

5. The method of any one of claims 1 to 4, wherein the cellular efflux transporter inhibitor is administered in an amount such that the bioavailability of the menin inhibitor is increased by at least 20%, 30%, 40%, 50%, 60%, 70%, 80%, 90%, 100%, 200%, 300%, or 400%.

6. The method of claim 4 or claim 5, wherein the bioavailability of the menin inhibitor is measured by Area Under the Curve (AUC) or Cₘₐₓ.

7. A method of increasing permeability of a menin inhibitor into cells (e.g., cancer cells) or increasing distribution of a menin inhibitor in a tissue (e.g., a cancer tissue, brain tissue, bone marrow, or plasma) in a subject comprising administering to the subject a menin inhibitor and a cellular efflux transporter inhibitor, optionally selected from a P-gp inhibitor and a BCRP inhibitor, wherein the subject optionally has a hematological malignancy.

8. The method of claim 7, wherein the permeability of the menin inhibitor through cells or the distribution of the menin inhibitor in a tissue is measured by the concentration of the menin inhibitor in the cells or in the tissue.

9. The method of any one of claims 1 to 8, wherein the cellular efflux transporter inhibitor is administered in an amount such that the permeability of the menin inhibitor through cells (e.g., cancer cells) or the distribution of the menin inhibitor in a tissue (e.g., cancer tissue, brain tissue, bone marrow, or plasma) is increased by at least 20%, 30%, 40%, 50%, 60%, 70%, 80%, 90%, 100%, 200%, 300%, or 400%.

10. The method of any one of claims 1 to 9, wherein the cellular efflux transporter inhibitor reduces efflux ratio of the menin inhibitor by at least 20%, 30%, 40%, 50%, 60%, 70%, 80%, or 90%.

11. The method of any one of claims 1 to 10, wherein the menin inhibitor and the cellular efflux transporter inhibitor are administered separately.

12. The method of any one of claims 1 to 11, wherein the menin inhibitor and the cellular efflux transporter inhibitor are administered by the same route.

13. The method of claim 12, wherein the menin inhibitor and the cellular efflux transporter inhibitor are each orally administered.

14. The method of any one of claims 1 to 13, wherein the menin inhibitor and the cellular efflux transporter inhibitor are sequentially administered.

15. The method of any one of claims 1 to 14, wherein the menin inhibitor and the cellular efflux transporter inhibitor are concurrently administered.

16. The method of any one of claims 1 to 15, wherein the hematological malignancy is selected from the group consisting of: Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute lymphocytic leukemia (ALL), mixed phenotype acute leukemia (MPAL), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma (CML), and chronic monocytic leukemia.

17. The method of claim 16, wherein the hematological malignancy is AML.

18. The method of any one of claims 1 to 17, wherein the hematological malignancy is relapsed or refractory.

19. The method of any one of claims 1 to 18, wherein the cellular efflux transporter inhibitor is a P-gp inhibitor.

20. The method of claim 19, wherein the P-gp inhibitor is selected from amiodarone, aldosterone, azithromycin, capmatinib, clarithromycin, cobcistat, daclatasvir, diosmin, dronedarone, elagolix, elacridar, eliglustat, flibanserin, fostamatinib, glecaprevir and/or pibrentasvir, ivacaftor, lapatinib, ledipasvir, neratinib, osimeritinib, propafenone, ranolazine, rolapitant, simeprevir, tepotinib, tezacaftor and/or ivacaftor, ticagrelor, tucatinib, velpatasvir, vemurafenib, voclosporin, lidocaine, clomiphene, cefoperazone, cortisol, ceftriaxone, dexamethasone, erythromycin, prednisone, ketoconazole, itraconazole, hydroxyitraconazole, posaconazole, progesterone, chloroquine, emetine, desipramine, quinidine, trazodone, hydroxychloroquine, dipyridamole, quinacrine, reserpine, quinine, cyclosporin A, bepridil, colchicine, diltiazem, FK-506, felodipine, quercetin, SDZ PSC-833, nifedipine, SDZ 280-446, nisoldipine, rifampin, terfenadine, nitrendipine, tumor necrosis factor, tiapamil, vitamin A, verapamil, etoposide, actinomycin D, daunorubicin, mitomycin-C, tamoxifen, taxol, RU-486, trimetrexase, devapamil, vinblastine, gallopamil, vincristine, emopamil, indinavir, nelfinavir, saquinavir, ritonavir, phenothiazines, and bupivacaine.

21. The method of claim 20, wherein the P-gp inhibitor is selected from ketoconazole, itraconazole, hydroxyitraconazole, and posaconazole.

22. The method of any one of claims 18 to 21, comprising administering to the subject a breast cancer resistance protein (BCRP) inhibitor.

23. The method of any one of claims 1 to 18, wherein the cellular efflux transporter inhibitor is a BCRP inhibitor.

24. The method of claim 22 or claim 23, wherein the BCRP inhibitor is selected from oteseconazole, biochanin A, chrysin, curcumin, eltrombopag, gefitinib, imatinib, Ko143, KS-176, fumitremorgin C (FTC), elacridar (GF120918), YHO-13351, diethylstilbestrol, cyclosporine A, prazosin, saquinavir, ritonavir, β-estradiol, verapamil, tamoxifen, triclabendazole sulfoxide, Hoechst 33342, PCI 29732, ML753286, CP-100356 hydrochloride, ML230, FD 12-9, quercetin, novobiocin, WK-X-34, tryprostatin A, quizartinib, omeprazole, methotrexate, ergocristine, nicardipine, ethinylestradiol, astemizole, telodipine, glibenclamide, ketoconazole, chlorprotixene, nitrendipine, chlorpromazine, progesterone, mifepristone, dipyridamole, lopinavir, amlodarone, simvastatin, loperamide, terfenadine, clotrimazol, spironolactone, maprotiline, digoxin, quinine, fexofenadine, diltiazem, erythromycin, etoposide, prednisone, trimethoprim, chlorzoxazone, folic acid, lansoprazole, dexlansoprazole, rantidine, cimetidine, indomethacin, prednisolone, propanolol, timolol, desipramine, pravastatin, hydrocortisone, sulfinpyrazone, fenolibrate, tipranavir, nelfinavir, erlotinib, flupentixol, celecoxib, thioridazine, isradipine, fendiline, medroxyprogesterone, pramoxine, piroxicam, terazosin, diazoxide, oxazepam, propafenone, tinidazole, meclizine, tetracycline, budesonide, desmethyldiazepam, nevirapine, diazepam, zanamivir, flurbiprofen, neomycin sulfate, nitrofurantoin, valacyclovir, carbamazepine, chenodeoxychloic acid, hydrochlorothiazide, amantadine, amoxicillin, phenytoin, antipyrine, bendroflumethiazide, ganciclovir, metoclopramide, pindolol, warfarin, amiloride, bupivacaine, carisoprodol, nizatidine, orphenadrine, procyclidine, acyclovir, atropine, captopril, furosemide, hydralazine, levothyroxine, salicyclic acid, sotalol, valganciclovir, levodopa, methimazole, sulindac, metoprolol, zidovudine, gliclazide, mesalazine, bupropion, and sulfasalazine.

25. The method of any one of claims 1 to 24, wherein the menin inhibitor is Compound I: 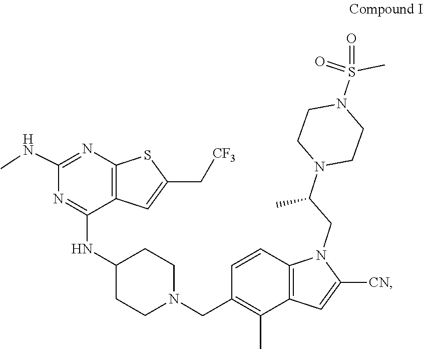
or a pharmaceutically acceptable form thereof.

26. The method of any one of claims 1 to 25, wherein administering the menin inhibitor comprises administering a therapeutically effective amount of the menin inhibitor.

27. A kit comprising: (1) a therapeutically effective amount of a pharmaceutical composition comprising a menin inhibitor and a pharmaceutically acceptable carrier or excipient, in a first dosage form; and (2) a composition comprising a cellular efflux transporter inhibitor, optionally selected from a P-gp inhibitor and a BCRP inhibitor, and a pharmaceutically acceptable carrier or excipient, in a second dosage form.

28. A pharmaceutical composition comprising 4-methyl-5-((4-((2-(methylamino)-6-((S)-2,2,2-trifluoro-1-hydroxyethyl)thieno[2,3-d]pyrimidin-4-yl)amino)piperidin-1-yl)methyl)-1-((S)-2-(4-(methylsulfonyl)piperazin-1-yl)propyl)-1H-indole-2-carbonitrile (Compound IB): 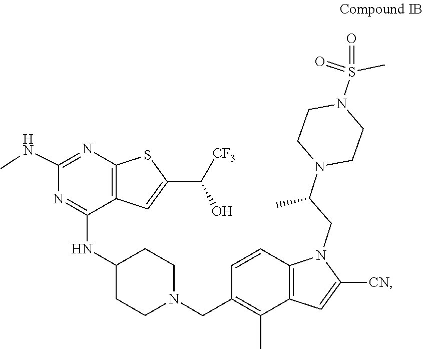
or a pharmaceutically acceptable form thereof,
and a pharmaceutically acceptable excipient or carrier.
